SEQUENCE LISTING (17 sequences)

SEQ ID NO: 1 - DNA, 1119 bp, Murinae gen. sp.
  CDS (19)..(642)
ggcctgactaccagaaacatggcgaccagcgctgttccaagtgaaaaccttcccacatataaactagtagtggtgggagatggtggtgtgggcaagagtgcgctcactattcagtttttccagaagatctttgtgcctgactacgaccccaccattgaagactcctacctgaagcatacagagattgacaatcagtgggccatcttggatgttctggacacagccgggcaggaggagttcagtgccatgcgggaacaatacatgcgcacaggggatggcttcctcattgtctactccgtcaccgacaaggccagcttcgagcacgtggaccgcttccaccagctcattctgcgtgtcaaggacagggagtcattcccaatgatcctcgtggccaacaaggtggatctgatgcacctaaggaaagtcaccagggaccaaggaaaagaaatggcaaccaaatacaatatcccatatatagagaccagtgccaaggacccgcctctcaacgtggataaaaccttccatgacctagttagagtaattaggcaacaggttccagagaaaaaccagaagaagaaaaagaagacaaaatggcgaggagacagggccaccggcactcacaaactgcagtgtgtcatcttgtgacagcctgaagccctgggcatagcaaccgtgaactgccagcccctgggaccagcccactgcctaactgcactgagaaccacttctaactacagcccttggctcttggactgggcattggaagggaatgagggaggagggggcagaagcaggccggggctggctttgctgcctgtcccaggagacagggctacagcttccaaaccttttgtgtgttgactgagcccagttcccagtctcttggtgggcttgtttcttttaactcataggctggtttgctatggaagtgcttacccacatacaacgcaccagacaagccatgagcaagcttcctccctgtcccatccccagtgtctgagctcttgtgtcttttgtagatttttaaattatttgagtaatgattattttattaaagaggtgtgtgcccattgcctgcgaagccccaagtctttggcagacctctgataaatgtctgca

SEQ ID NO: 2 - protein, 208 aa, Murinae gen. sp.
MATSAVPSENLPTYKLVVVGDGGVGKSALTIQFFQKIFVPDYDPTIEDSYLKHTEIDNQWAILDVLDTAGQEEFSAMREQYMRTGDGFLIVYSVTDKASFEHVDRFHQLILRVKDRESFPMILVANKVDLMHLRKVTRDQGKEMATKYNIPYIETSAKDPPLNVDKTFHDLVRVIRQQVPEKNQKKKKKTKWRGDRATGTHKLQCVIL

SEQ ID NO: 3 - DNA, 1085 bp, Homo sapiens
  CDS (102)..(725)
cggcggcgacgctgcctcctcaccggcgcaggctaggagggggcggcctgagtgccgtagccgagccggggctggagcgcgcggtctgacctacgagaaacatggcgaccagcgccgtccccagtgacaacctccccacatacaagctggtggtggtgggggatgggggtgtgggcaaaagtgccctcaccatccagtttttccagaagatctttgtgcctgactatgaccccaccattgaagactcctacctgaaacatacggagattgacaatcaatgggccatcttggacgttctggacacagctgggcaggaggaattcagcgccatgcgggagcaatacatgcgcacgggggatggcttcctcatcgtctactccgtcactgacaaggccagctttgagcacgtggaccgcttccaccagcttatcctgcgcgtcaaagacagggagtcattcccgatgatcctcgtggccaacaaggtcgatttgatgcacttgaggaagatcaccagggagcaaggaaaagaaatggcgaccaaacacaatattccgtacatagaaaccagtgccaaggacccacctctcaatgtcgacaaagccttccatgacctcgttagagtaattaggcaacagattccggaaaaaagccagaagaagaagaagaaaaccaaatggcggggagaccgggccacaggcacccacaaactgcaatgtgtgatcttgtgaggcctgcaggcctgaaggcctcgggcacagtgacggtggcctggccagccctcgggacccctccccacctaactgcactgaaaccatttctaaccacaacccttggcccaaggacttggtacaggaagggagaagggcaggtgggcagggagcaagacagggtctggcttttgccaagaggaacggggctttttccaccttcttcaaaagagacaagggaaggccacctgttaaagcaggaagcagcatcaagttgccccttgggcccccccatgttgtttggatttcaaaccgggtttccttcccccttcttttcgggttgggtgttgttggttatttggttaactacatatggtt

SEQ ID NO: 4 - protein, 208 aa, Homo sapiens [sequence omitted]

SEQ ID NO: 5 - protein, 19 aa, Homo sapiens
CKKKTKWRGDRATGTHKLQ

SEQ ID NO: 6 - DNA, 24 bp, Murinae gen. sp.
agcactctccagcctctcaccgca

SEQ ID NO: 7 - DNA, 12 bp, Murinae gen. sp.
gatctgcggtga

SEQ ID NO: 8 - DNA, 24 bp, Murinae gen. sp.
accgacgtcgactatccatgaaca

SEQ ID NO: 9 - DNA, 12 bp, Murinae gen. sp.
gatctgttcatg

SEQ ID NO: 10 - DNA, 24 bp, Murinae gen. sp.
aggcaactgtgctatccgagggaa

SEQ ID NO: 11 - DNA, 12 bp, Murinae gen. sp.
gatcttccctcg

SEQ ID NO: 12 - DNA, 18 bp, Murinae gen. sp.
ccagactggcacagttcc

SEQ ID NO: 13 - DNA, 19 bp, Murinae gen. sp.
tgctgtagaagccgaagcc

SEQ ID NO: 14 - DNA, 18 bp, Homo sapiens
gaattcagcgccatgcgc

SEQ ID NO: 15 - DNA, 20 bp, Homo sapiens
cctcacaagatcacacattg

SEQ ID NO: 16 - protein, 189 aa, p21 H-Ras
MTEYKLVVVGAGGVGKSALTIQLIQNHFVDEYDPTIEDSYRKQVVIDGETCLLDYLDTAGQEEYSAMRDQYMRYGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 17 - protein, 218 aa, R-Ras
MSSGAASGTGRGRPRGGGPGPRDPPPGETHKLVVVGGGGVGKSALTIQFIQSYFVSDYDPTIEDSYTKICTVDGIPARLDILDTAGQEEFGAMREQYMRAGNGFLLVFAINDRQSFNEVGKLFTQILRVKDRDDFPIVLVGNKADLENQRQVLRSEASSFSASHHMTYFEASAKLRLNVDEAFEQLVRAVRKYQEQELPPSPPSAPRKKDGGCPCVLL

(19) United States
(12) Patent Application Publication
(10) Pub. No.: US 2002/0156013 A1
(43) Pub. Date: Oct. 24, 2002

(54) Asthma associated factors as targets for treating atopic allergies including asthma and related disorders

(76) Inventors: Jean-Christophe Renauld, Krainem (BE); Jamila Louahed, East Norristown, PA (US); Luigi Grasso, Philadelphia, PA (US); Roy Levitt, Ambler, PA (US); Nicholas Nicolaides, Media, PA (US)

Correspondence Address: Mary Anne Schofield, FULBRIGHT & JAWORSKI L.L.P., Market Square, 801 Pennsylvania Avenue, N.W., Washington, DC, 20004-2615, US

(21) Appl. No.: 10/067,813

(22) Filed: Feb. 8, 2002

Related U.S. Application Data
(63) Continuation of application No. 09/157,247, filed on Sep. 18, 1998, now abandoned.
(60) Provisional application No. 60/059,509, filed on Sep. 19, 1997.

(51) Int. Cl. A61K 38/17; C12P 21/02; C12N 5/06; A61K 31/56; C12N 9/10; A61K 31/366; A61K 31/225

(52) U.S. Cl. 514/12; 536/23.2; 435/193; 435/69.1; 435/325; 435/320.1; 514/169; 514/460; 514/548

(57) ABSTRACT

A new gene in the Ras family that is induced by IL-9, thereby providing a therapeutic target in IL-9 mediated development of atopic allergy, asthma-related disorders and certain lymphomas or leukemias. A method for the identification and use of small molecule inhibitors of Ras to treat these disorders. A method for diagnosing susceptibility to, and assessing treatment of atopic allergy, asthma-related disorders and certain lymphomas and leukemias by measuring the level of Ras in biologic samples using antibody specific for the Ras protein.

### CROSS-REFERENCE TO RELATED APPLICATION

This application claims the benefit of U.S. Provisional Patent Application Serial No. 60/059,509 which was filed Sep. 19, 1997. This invention is also related to the subject matter of U.S. patent application Ser. Nos. 08/697,419; 08/697,360; 08/697,473; 08/697,472; 08/697,471; 08/702,105; 08/702,110; 08/702,168; and 08/697,440, filed on Aug. 23, 1996 and Ser. No. 08/874,503 filed on Jun. 13, 1997 all of which are incorporated herein by reference. In addition, this application is related to U.S. patent application Ser. No. 08/980,872 which was filed Dec. 1, 1997 and which is incorporated herein by reference.

### FIELD OF THE INVENTION

This invention relates to modulating activities associated with the IL-9 pathway for the treatment of atopic allergies and related disorders like asthma. It also relates to inhibition of the IL-9 pathway for the treatment of cancer.

### BACKGROUND OF THE INVENTION

Inflammation is a complex process in which the body's defense system combats foreign entities. While the battle against foreign entities may be necessary for the body's survival, some defense systems respond to foreign entities, even innocuous ones, as dangerous and thereby damage surrounding tissue in the ensuing battle. Atopic allergy, or atopy, is an ecogenetic disorder, where genetic background dictates the response to environmental stimuli, such as pollen, food, dander and insect venoms. The disorder is generally characterized by an increased ability of lymphocytes to produce IgE antibodies in response to ubiquitous antigens. Activation of the immune system by these antigens leads to allergic inflammation and may occur after ingestion, penetration through the skin or after inhalation. When this immune activation occurs and is accompanied by pulmonary inflammation and bronchial hyperresponsiveness, this disorder is broadly characterized as asthma. Certain cells are critical to this inflammatory reaction and they include T cells and antigen-presenting cells, B cells that produce IgE, and basophils and eosinophils that bind IgE. These inflammatory cells accumulate at the site of allergic inflammation and the toxic products they release contribute to tissue destruction related to these disorders.

While asthma is generally defined as an inflammatory disorder of the airways, clinical symptoms arise from intermittent air flow obstruction. It is a chronic, disabling disorder that appears to be increasing in prevalence and severity (Gergen et al., 1992). It is estimated that 30-40% of the population suffer with atopic allergy and 15% of children and 5% of adults in the population suffer from asthma (Gergen et al., 1992). Thus, an enormous burden is placed on our health-care resources.

Interestingly, while most individuals experience similar environmental exposures, only certain individuals develop atopic allergy and asthma. This hypersensitivity to environmental allergens known as atopy, is often indicated by elevated serum IgE levels or abnormally intense skin test response to allergens in atopic individuals as compared to non-atopics (Marsh et al., 1982). Strong evidence for a close relationship between atopic allergy and asthma is derived from the fact that most asthmatics have clinical and serologic evidence of atopy (Clifford et al., 1987; Gergen, 1991; Burrows et al., 1992; Johannson et al., 1972; Sears et al., 1991; Halonen et al., 1992). In particular, younger asthmatics have a high incidence of atopy (Marsh et al., 1982). In addition, immunologic factors associated with an increase in total serum IgE levels are very closely related to impaired pulmonary function (Burrows et al., 1989).

Both the diagnosis and treatment of these disorders are problematic (Gergen et al., 1992). The assessment of inflamed lung tissue is often difficult and frequently the source of the inflammation cannot be determined. Without knowledge of the source of the airway inflammation and protection from the inciting foreign environmental agent or agents, the inflammatory process cannot be interrupted. It is now generally accepted that failure to control pulmonary inflammation leads to significant loss of lung function over time.

Current treatments suffer their own set of disadvantages. The main therapeutic agents, β agonists, reduce the symptoms thereby transiently improving pulmonary function, but do not affect the underlying inflammation so that lung tissue remains in jeopardy. In addition, constant use of β agonists results in desensitization which reduces their efficacy and safety (Molinoff et al., 1995). The agents that can diminish the underlying inflammation, the anti-inflammatory steroids, have their own list of disadvantages that range from immunosuppression to bone loss (Molinoff et al., 1995).

Because of the problems associated with conventional therapies, alternative treatment strategies have been evaluated. Glycophorin A (Chu et al., 1992), cyclosporin (Alexander et al., 1992; Morely, 1992) and a nonapeptide fragment of interleukin 2 (IL-2) (Zavyalov et al., 1992) all inhibit potentially critical immune functions associated with homeostasis. What is needed in the art is a treatment for asthma that addresses the underlying pathogenesis. Moreover, these therapies must address the episodic nature of the disorder and the close association with allergy and intervene at a point downstream from critical immune functions.

In the related patent applications mentioned above, it was demonstrated that interleukin 9 (IL-9), its receptor and activities effected by IL-9 are the appropriate targets for therapeutic intervention in atopic allergy, asthma and related disorders. Applicants now disclose related genes that are important in atopic allergy, asthma and certain lymphomas as well as methods of regulating these genes for therapeutic intervention.

Mediator release from mast cells by allergen has long been considered a critical initiating event in allergy. IL-9 was originally identified as a mast cell growth factor (Schmitt et al., 1989) and applicants have previously demonstrated that IL-9 appears to up-regulate the expression of mast cell proteases including MCP-1, MCP-2, MCP4 (Godfraind et al., 1998) and granzyme B (Louahed et al., 1995). Thus, IL-9 may serve a role in the proliferation and differentiation of mast cells. Moreover, IL-9 up-regulates the expression of the alpha chain of the high affinity IgE receptor (Louahed et al., 1995). Elevated IgE levels are considered to be a hallmark of atopic allergy and a risk factor for asthma. Furthermore, both in vitro and in vivo studies have shown IL-9 to potentiate the release of IgE from primed B cells (Dugas et al., 1993; Petit-Frere et al., 1993).

Based on the data presented in the related patents listed above, there is substantial support for the IL-9 gene candidate in asthma. First, applicants demonstrate linkage homology between humans and mice, suggesting the same gene is responsible for producing biologic variability in response to antigen in both species. Second, differences in expression of the murine IL-9 candidate gene were associated with biologic variability in bronchial responsiveness. In particular, a loss of function is associated with a lower baseline bronchial response in C57BL6 mice. Third, recent evidence for linkage disequilibrium in data from humans suggests IL-9 may be associated with atopy and bronchial hyperresponsiveness consistent with a role for this gene in both species (Doull et al., 1996). Moreover, applicants have demonstrated that a genetic alteration in the human gene appears to be associated with loss of cytokine function and lower IgE levels. Fourth, the pleiotropic functions of this cytokine and its receptor in the allergic immune response strongly support a role for the IL-9 pathway in the complex pathogenesis of asthma. Fifth, in humans, biologic variability in the IL-9 receptor also appears to be associated with atopic allergy and asthma. Finally, despite the inherited loss of IL-9 receptor function, these individuals appear to be otherwise healthy. Thus, nature has demonstrated in atopic individuals that the therapeutic down-regulation of IL-9 and IL-9 receptor genes or genes activated by IL-9 and its receptor is likely to be safe.

Of equal importance to the relationship of IL-9 with atopic disorders is its connection to cell proliferation and differentiation. !L-9 was also initially characterized for its ability to promote growth of T helper cells (Uyttenhove et al., 1988). Subsequently, several other activities were attributed to IL-9 including: differentiation of hematopoietic and neuronal progenitor cells and proliferation as well as differentiation of mast cells (Renauld et al., 1995). In addition, there is some evidence for involvement of IL-9 in both human and murine tumorigenesis (Vink et al., 1993). Overexpression of IL-9 has been associated with a high susceptibility to T cell lymphomas in vivo and an autocrine IL-9 loop has been characterized in some human Hodgkin lymphomas (Renauld et al., 1994; Merz et al., 1991). It therefore seems likely that IL-9 is also involved in other neoplasms of T cell origin including T cell leukemias and Mycosis fungoides.

Applicants have demonstrated that activity of Ras proteins can be regulated not only at the level of GTPase activity but also at the mRNA level in an IL-9 dependent manner. The Ras superfamily of oncogenes play a major role in many signal transduction pathways that lead to cell growth and differentiation (Quinn et al., 1993) and are associated with tumorigenesis. Cell proliferation is primarily mediated by the H-Ras, K-Ras, N-Ras and R-Ras subfamily. These small GTPases have a high degree of homology at the amino acid level including five well conserved amino acid motifs involved in guanine nucleotide binding and hydrolysis (Bourne et al., 1991). Upon GTP binding, they activate the serine/threonine kinase c-Raf-1, which in turn, activates the mitogen-activated protein kinase kinase (MEK), which finally activates the mitogen-activated protein kinase (MAPK) (Waskiewicz et al., 1995). Despite this similarity in structure and function, differences are found in regulation of GTPase activity through interaction with various downstream effector proteins suggesting that each Ras-related protein plays a distinct regulatory role in vivo (Marshall, 1996).

Thus, the art now understands how the IL-9 gene, its receptor and how their functions are related to atopic allergy, asthma, cell proliferation, transformation and tumorigenesis. Therefore, a specific need in the art exists for elucidation of the role of genes which are regulated by IL-9 in the etiology of these disorders. Furthermore, most significantly, based on this knowledge, there is a need for the identification of agents that are capable of regulating the activity of these genes or their gene products for treating these disorders.

### SUMMARY OF THE INVENTION

Applicants have identified a new gene from the Ras family of oncogenes designated M-Ras. This gene is selectively up-regulated by IL-9 and therefore part of the IL-9 signaling pathway.

In a first embodiment, the invention provides purified and isolated DNA molecules having nucleotide sequences encoding human M-Ras or functionally effective fragments thereof.

The invention further provides purified and isolated protein molecules having amino acid sequences comprising human M-Ras or functionally effective fragments thereof.

In a second embodiment, the invention provides purified and isolated DNA molecules having nucleotide sequences encoding murine M-Ras or functionally effective fragments thereof.

The invention further provides purified and isolated protein molecules having amino acid sequences comprising murine M-Ras or functionally effective fragments thereof.

Applicants have satisfied the need for diagnosis and treatment of atopic allergy, asthma and certain lymphomas or leukemias by demonstrating the role of M-Ras in the pathogenesis of these disorders. Therapies for these disorders are derived from the down-regulation of M-Ras as a member of the IL-9 pathway.

The identification of M-Ras has led to the discovery of compounds capable of down-regulating its activity. Molecules that down-regulate M-Ras are therefore claimed in the invention. Down-regulation is defined here as a decrease in activation, function or synthesis of M-Ras, its ligands or activators. It is further defined as a increase in degradation of M-Ras, its ligands or activators. Down-regulation is therefore achieved in a number of ways. For example, by administration of molecules that can destabilize binding of M-Ras with its ligands. Such molecules encompass polypeptide products, including those encoded by the DNA sequences of the M-Ras gene or DNA sequences containing various mutations of this gene. These mutations may be point mutations, insertions, deletions or spliced variants of the M-Ras gene. This invention also includes truncated polypeptides encoded by the DNA molecules described above. These polypeptides being capable of interfering with the interaction of M-Ras with its ligands and other proteins.

A further embodiment of this invention includes the down-regulation of M-Ras function by altering expression of the M-Ras gene, the use of antisense therapy being an example. Down-regulation of M-Ras expression is accomplished by administering an effective amount of antisense oligonucleotide. These antisense molecules can be fashioned from the DNA sequence of the M-Ras gene or sequences containing various mutations, deletions, insertions or spliced variants. Another embodiment of this invention relates to the use of isolated RNA or DNA sequences derived from the M-Ras gene. These sequences contain various mutations such as point mutations, insertions, deletions or spliced variant mutations of the M-Ras gene and can be useful in gene therapy.

The structure of M-Ras has been examined and analyzed in great detail and amino acid residues of M-Ras critical for activation or binding to ligands have been identified. These sites include but are not limited to residues 20-27 comprising the ATP/GTP binding site, residues 193-195 comprising a RGD sequence likely involved in adhesion to cell structural proteins and residues 205-208 which represent the site for farnesyl transferase or geranylgeranyl transferase activation. Farnesyl transferase and geranylgeranyl transferase inhibitors are well known in the art and examples of such inhibitors have been previously described (Qian et al., 1997). The use of such inhibitors for down-regulation of M-Ras are within the claimed invention.

This invention further includes small molecules with the necessary three-dimensional structure required to bind with sufficient affinity to block the interaction of M-Ras with its ligands. M-Ras blockade, resulting in down-regulation of M-Ras activity, calcium flux and other processes of proinflammatory cells where it is expressed, make these molecules useful in treating inflammation associated with atopic allergy, asthma and related disorders. M-Ras blockade by these same molecules make them useful for the treatment of certain lymphomas or leukemias as well.

In a further embodiment, aminosterol compounds are demonstrated to block M-Ras induction by IL-9 or antigen and therefore are useful in treating atopic allergies, asthma and certain lymphomas or leukemias. In yet another embodiment, inhibitors that block activation pathways downstream from M-Ras are shown to down-regulate the IL-9 pathways and therefore can also be used for the treatment of atopic allergies, asthma and certain lymphomas or leukemias.

The products discussed above represent various effective therapeutic agents in treating atopic allergies, asthma and certain lymphomas or leukemias. Applicants have provided antagonists and methods for identifying antagonists that are capable of down-regulating M-Ras. Applicants also provide methods for down-regulating M-Ras activity by administering truncated protein products, aminosterols or the like.

Applicants also provide a method for the diagnosis of susceptibility to atopic allergy, asthma and certain lymphomas or leukemias by describing a method for assaying the induction of M-Ras, its functions or downstream activities. In a further embodiment, applicants provide methods to monitor the effects of M-Ras down-regulation as a means to follow the treatment of atopic allergy, asthma and certain lymphomas or leukemias.

The accompanying figures, which are incorporated in and constitute a part of this specification, illustrate several embodiments of the invention and together with the description, serve to explain the principle of the invention.

### BRIEF DESCRIPTION OF THE FIGURES

FIG. 1: Nucleotide sequence of the murine M-Ras cDNA (SEQ ID NO:1) and translated amino acid sequence (SEQ ID NO:2).

FIG. 2: Alignment of the murine M-Ras protein with p21H-Ras and R-Ras.

FIG. 3: Kinetics of M-Ras expression induced by IL-9.

FIG. 4: M-Ras expression in normal mice (FVB) compared to transgenic mice overexpressing the IL-9 gene (Tg5).

FIG. 5: Nucleotide sequence of the human M-Ras cDNA (SEQ ID NO:3) and translated amino acid sequence (SEQ ID NO:4).

FIG. 6: Expression of M-Ras in human tissues standardized with the level of β-actin.

FIG. 7: M-Ras induction by IL-9 (25 ng/ml) in human K562 cells.

FIG. 8: Mutants of the murine M-Ras gene.

FIG. 9: Effect of murine M-Ras mutation on the proliferation of BaF3 cells.

FIG. 10: Transformation of NIH3T3 cells by constitutively active M-Ras.

FIG. 11: Summary of the PathDetect Method.

FIG. 12: Activation of the MAPK pathway by activated M-Ras.

FIG. 13: Effect of the MAPK inhibitor PD98059 on M-Ras-2 activity.

FIG. 14: Activation of M-Ras abrogates dexamethasone induced apoptosis.

FIG. 15: Selective blocking of IL-9 signaling via the M-Ras pathway as opposed to IL-2 signaling pathway using MAPK inhibitor PD98059.

FIG. 16: Specific blocking of the M-Ras signaling pathway by PD98059 independent of cell type.

FIG. 17: Specific blocking of IL-9 signaling pathway by the p38 MAPK inhibitor SB202190.

FIG. 18: Effect of manumycin A on IL-9 induced proliferation of RA3 cells.

FIG. 19: Effect of lovastatin on IL-9 induced proliferation of TS2 cells.

FIG. 20: Structure of some aminosterols tested as inhibitors of M-Ras induction.

FIG. 21: Inhibition of M-Ras induction by aminosterols.

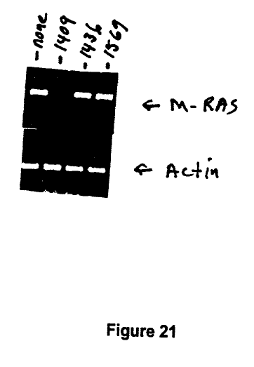

FIG. 22: Inhibition of M-Ras prenylation by lovastatin.

### DETAILED DESCRIPTION OF THE INVENTION

Applicants have resolved the needs in the art by elucidating a gene in the IL-9 pathway, herein referred to as M-Ras, and identifying compositions affecting that gene or gene product which may be used in the diagnosis, prevention or treatment of atopic allergy including asthma and related disorders. Asthma encompasses inflammatory disorders of the airways with reversible airflow obstruction. Atopic allergy refers to atopy and related disorders including asthma, bronchial hyperresponsiveness, rhinitis, urticaria, allergic inflammatory disorders of the bowel and various forms of eczema. Atopy is a hypersensitivity to environmental allergens expressed as the elevation of total serum IgE or abnormal skin test responses to allergens as compared to controls. Bronchial hyperresponsiveness is defined here as a heightened bronchoconstrictor response to a variety of stimuli.

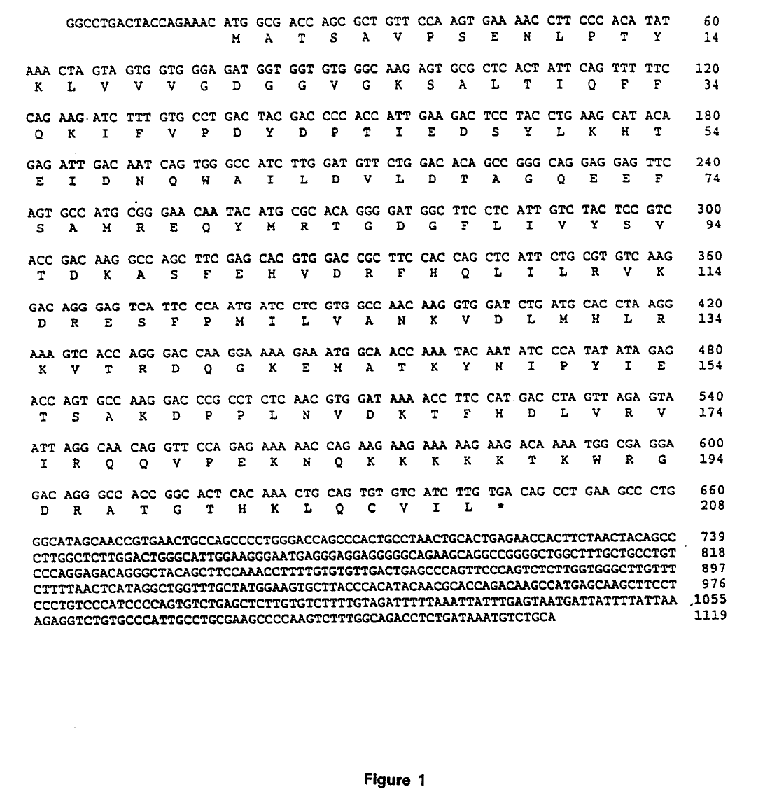

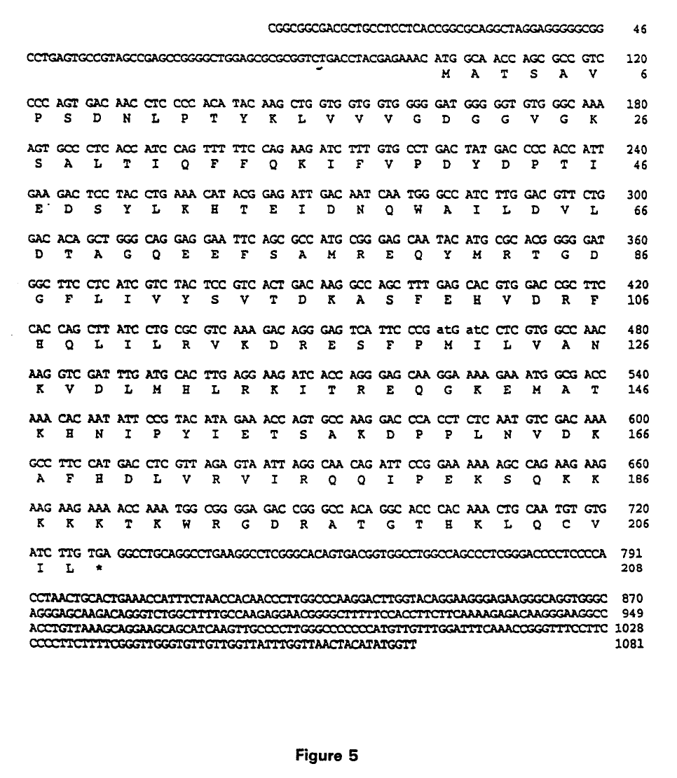

Accordingly, the invention provides a purified and isolated nucleic acid molecule comprising a nucleotide sequence encoding murine (FIG. 1) or human (FIG. 5) M-Ras or a fragment thereof. The invention also includes degenerate sequences of the DNA as well as sequences that are substantially homologous. The exemplified source of the M-Ras for the invention is murine and human, although M-Ras-from any source is encompassed by the invention. The nucleic acid molecule or fragment thereof, may be synthesized using methods known in the art. It is also possible to produce the molecule by genetic engineering techniques, by constructing DNA using any accepted technique, cloning the DNA in an expression vehicle and transfecting the vehicle into a cell which will express the compound. See, for example, the methods set forth in Sambrook et al., Molecular Cloning: A Laboratory Manual, 2nd edition, Cold Spring Harbor Laboratory Press, 1985.

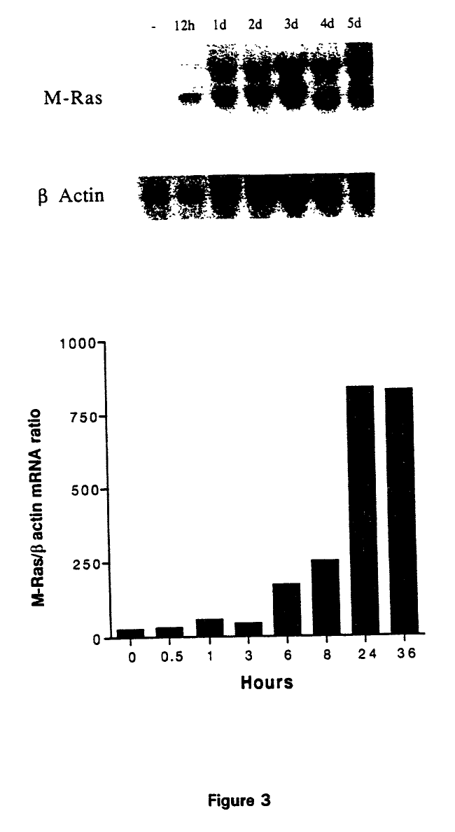

The M-Ras gene was identified by subtractive cDNA cloning experiments performed in order to identify genes specifically induced by IL-9 as described in Example 1. Applicants used a murine T lymphocyte cell clone (TS2) that can grow in the presence of either IL-9 or IL-2 and isolated genes expressed when cells were stimulated by IL-9. The kinetics of M-Ras expression after stimulation with IL-9 was also studied on TS2 cells grown in the presence of IL-2 and stimulated with IL-9 for various periods of time before RNA extraction and Northern Blot analysis with M-Ras cDNA. As shown in FIG. 3, murine M-Ras expression increased after six hours, reaching maximal levels after 24 hours, suggesting a rapid and prolonged process promoted in response to IL-9. Similar results were obtained when M-Ras expression was also assessed by RT-PCR and quantified by standardization with the level of M-actin expression.

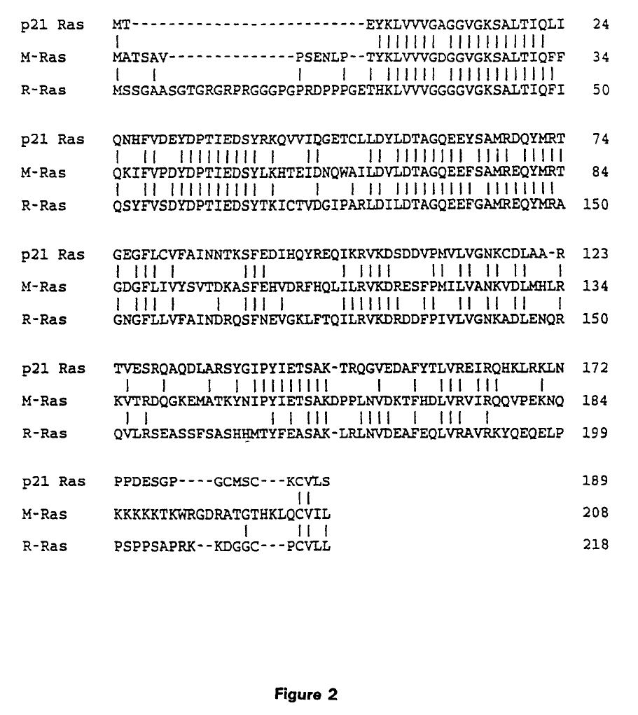

The murine M-Ras gene displayed significant homology (-50%) with members of the Ras family of oncogenes, particularly with H-Ras (57%), N-Ras (58%) and R-Ras (50%) (FIG. 2). A full length cDNA was cloned from a murine cDNA library and the human cDNA was cloned by PCR based on the murine sequence, as well as by screening a human library with a human cDNA probe. As expected for members of this family, some motifs involved in GTP binding are well conserved. The C-terminus is also typical for these proteins, with the presence of a prenylation site: CAAX, where C is a cysteine, A is a hydrophobic residue and X can be any residue. A Japanese group has introduced into the GenBank database, a rat cDNA sequence corresponding to a new member of the Ras family. This gene was cloned from a rat brain cDNA library and was designated M-Ras, apparently because it was expressed at high level in muscle tissue. The rat cDNA sequence displays significant homology with the human (90%) and murine (96%) sequences described herein.

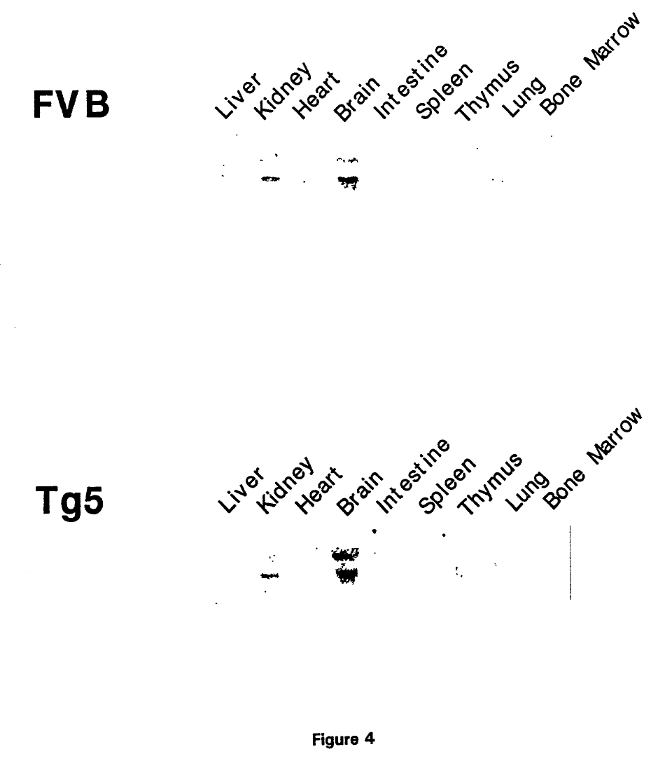

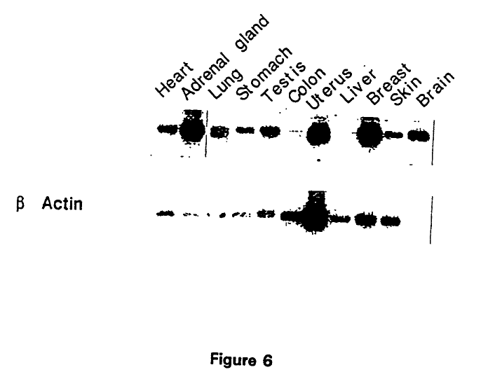

Expression of M-Ras appears to be ubiquitous in the human (FIG. 6) with maximal levels in adrenal gland, lung, breast and brain while in the mouse it is found primarily in the brain and kidney. No increased expression was detected in a panel of tissues from IL-9 transgenic mice (FIG. 4), suggesting that this gene is not induced by IL-9 in every tissue. However, M-Ras gene induction by IL-9 was observed in murine T helper cell clones, but not in IL-9 responsive mast cells. Thus, IL-9 induced the expression of this gene in only a subtype of IL-9 responsive cells.

Nucleic acid molecules of the invention include polynucleotides encoding murine and human M-Ras with the sequences of FIG. 1 (SEQ ID NO:1) and FIG. 5 (SEQ ID NO:3), respectively, as well as all nucleic acid sequences complementary to these sequences. A complementary sequence may include an antisense nucleotide.

It is understood that all polynucleotides encoding all or a portion of M-Ras are also included herein, as long as they encode a polypeptide with the functional activities of M-Ras as set forth herein. Polynucleotide sequences of the invention include DNA, cDNA, synthetic DNA and RNA sequences which encode M-Ras. Such polynucleotides also include naturally occurring, synthetic and intentionally manipulated polynucleotides. For example, such polynucleotide sequences may comprise genomic DNA which may or may not include naturally occurring introns. Moreover, such genomic DNA may be obtained in association with promoter regions or poly A sequences. As another example, portions of the mRNA sequence may be altered due to alternate RNA splicing patterns or the use of alternate promoters for RNA transcription. As yet another example, M-Ras polynucleotides may be subjected to site-directed mutagenesis.

The polynucleotides of the invention further include sequences that are degenerate as a result of the genetic code. The genetic code is said to be degenerate because more than one nucleotide triplet codes for the same amino acid. There are 20 natural amino acids, most of which are specified by more than one codon. It will be appreciated by those skilled in the art that as a result of the degeneracy of the genetic code, a multitude of nucleotide sequences, some bearing minimal nucleotide sequence homology to the nucleotide sequences of SEQ ID NO:1 and SEQ ID NO:3 may be produced as a result of this invention. Therefore, all degenerate nucleotide sequences are included in the invention as long as the amino acid sequence of the M-Ras polypeptide encoded by the nucleotide sequence is functionally unchanged or substantially similar in function. The invention specifically contemplated each and every possible variation of peptide or nucleotide sequence that could be made by selecting combinations based on the possible amino acid and codon choices made in accordance with the standard triplet genetic code as applied to the sequences of SEQ ID NO:1 and SEQ ID NO:3 and all such variations are to be considered specifically disclosed herein.

Also included in the invention are fragments (portions, segments) of the sequences disclosed herein which selectively hybridize to the sequences of SEQ ID NO:1 and SEQ ID NO:3. Selective hybridization as used herein refers to hybridization under stringent conditions (See, for example, the techniques in Maniatis et al., Molecular Cloning: A Laboratory Manual, Cold Spring Harbor Laboratory Press, 1989), which distinguishes related from unrelated nucleotide sequences. The active fragments of the invention, which are complementary to mRNA and the coding strand of DNA, are usually at least about 15 nucleotides, more usually at least 20 nucleotides, preferably 30 nucleotides and more preferably may be 50 nucleotides or more.

As used herein, “stringent conditions” are conditions in which hybridization yields a clear and readable sequence. Stringent conditions are those that (1) employ low ionic strength and high temperature for washing, for example, 0.015 M NaCl, 0.0015 M sodium citrate, 0.1% SDS buffer at 50° C., or (2) employ during hybridization a denaturing agent such as formamide, for example, 50% fornamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C. Another example is using 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS and 10% dextran sulfate at 42° C., with washes at 42° C. in 0.2× SSC and 0.1% SDS. A skilled artisan can readily determine and vary the stringency conditions appropriately to obtain a clear and detectable hybridization signal.

The present invention provides nucleic acid molecules encoding M-Ras proteins which hybridize with nucleic acid molecules comprising sequences complementary to either SEQ ID NO:1 or to SEQ ID NO:3 under conditions of sufficient stringency to produce a clear signal. As used herein, “nucleic acid” is defined as RNA or DNA encoding M-Ras peptides, nucleic acid molecules complementary to nucleic acids encoding such peptides, nucleic acid molecules which hybridize to such nucleic acids and remain stably bound to them under stringent conditions, nucleic acid molecules which encode polypeptides sharing at least 60% sequence identity, preferably at least 75% sequence identity, and more preferably at least 80% sequence identity with the M-Ras peptide sequences or nucleic acid molecules which comprise nucleotide sequences sharing at least 60% or 70% sequence identity with the open-reading-frame of SEQ ID NO:1 or to SEQ ID NO:3, preferably 80% or 85% sequence identity with the open-reading-frame of SEQ ID NO:1 or to SEQ ID NO:3, or more preferably, 90%, 91%, 95% or 97% sequence identity with the open-reading-frame of SEQ ID NO:1 or to SEQ ID NO:3. Nucleic acid molecules of the invention may be operably linked to any available vector, such as expression vectors. The resulting vectors may then be transformed or transfected into appropriate host cells (see Kriegler, Gene Transfer and Expression, Stockton Press, 1990).

Homology or sequence identity is determined by BLAST (Basic Local Alignment Search Tool) analysis using the algorithm employed by the programs blastp, blastn, blastx, tblastn and tblastx (Karlin , et al. Proc. Natl. Acad. Sci. USA 87: 2264-2268 (1990) and Altschul, S. F., J. Mol. Evol. 36: 290-300(1993), fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff, et al. Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively.

The invention further provides substantially pure M-Ras polypeptides. The term “substantially pure” as used herein refers to M-Ras polypeptide which is substantially free of other proteins, lipids, carbohydrates or other materials with which it is naturally associated. One skilled in the art can purify M-Ras using standard techniques for protein purification (Matsumoto et al., 1997; Self et al., 1995).

The invention also provides amino acid sequences coding for murine M-Ras polypeptides (SEQ ID NO:2) and human M-Ras polypeptides (SEQ ID NO:4). The polypeptides of the invention include those which differ from SEQ ID NO:2 and SEQ ID NO:4 as a result of conservative variations. The terms “conservative variation” or “conservative substitution” as used herein denotes the replacement of an amino acid residue by another, biologically similar residue. Conservative variations or substitutions are not likely to change the shape of the polypeptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine, and the like. Therefore, all conservative substitutions are included in the invention as long as the M-Ras polypeptide encoded by the nucleotide-sequence is functionally unchanged or similar.

As used herein, an isolated M-Ras protein can be a full-length M-Ras protein or any homologue of such a protein, such as a M-Ras protein in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycosylphosphatidyl inositol), wherein modified protein retains the physiological characteristics of natural M-Ras. A homologue of a M-Ras protein is a protein having an amino acid sequence that is sufficiently similar to a natural M-Ras protein amino acid sequence that a nucleic acid sequence encoding the homologue is capable of hybridizing under stringent conditions to (i.e., with) a nucleic acid sequence encoding the natural M-Ras protein amino acid sequence. Appropriate stringency requirements are discussed above.

M-Ras protein homologues can be the result of allelic variation of a natural gene encoding a M-Ras protein. A natural gene refers to the form of the gene found most often in nature. M-Ras protein homologues can be produced using techniques known in the art including, but not limited to, direct modifications to a gene encoding a protein using, for example, classic or recombinant DNA techniques to effect random or targeted mutagenesis.

Minor modifications of the M-Ras primary amino acid sequence may result in proteins which have substantially equivalent activity as compared to the M-Ras polypeptides described herein in SEQ ID NO:2 and SEQ ID NO:4. As used herein, a “functional equivalent” of the M-Ras protein is a protein which possesses a biological activity or immunological characteristic substantially similar to a biological activity or immunological characteristic of non-recombinant, or natural, M-Ras. The term “functional equivalent” is intended to include the fragments, variants, analogues, homologues, or chemical derivatives of a molecule which possess the biological activity of the M-Ras proteins of the present invention.

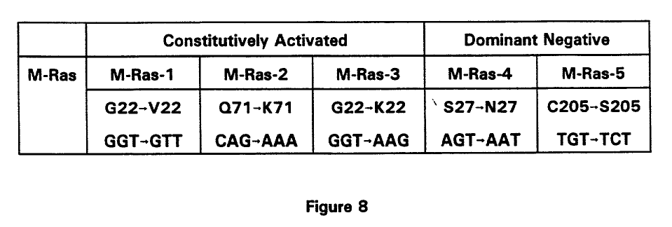

Ras proteins are known to be active when they are bound to a molecule of GTP and inactive when GTP is hydrolyzed to GDP. In order to characterize the activity of M-Ras, applicants generated mutant forms of the murine protein that, based on mutations of H-Ras, should correspond to either constitutively activated forms or dominant negative forms. These mutants are presented in FIG. 8.

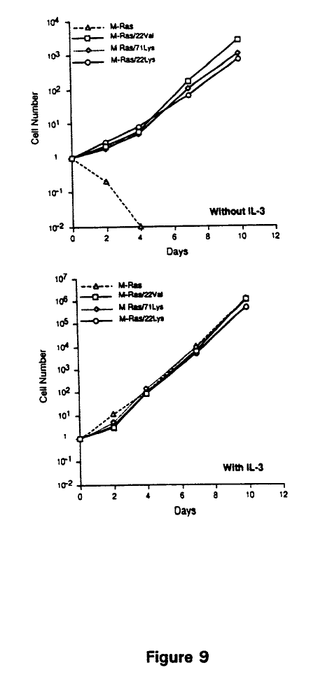

To test the oncogenic potential of these mutants, applicants transfected them into BaF3, a murine B cell line that depends on IL-3 for its proliferation. The proliferation of the transfectants was analyzed in the presence or absence of IL-3. The results of this experiment are shown in FIG. 9. Cells transfected with the wild type M-Ras (top panel) die rapidly in the absence of IL-3, exactly as the parental BaF3 cells, indicating that overexpression of M-Ras does not affect survival or proliferation. The same result was seen with M-Ras4 and M-Ras-5, which were supposed to correspond to dominant negative mutants. By contrast, when BaF3 cells were transfected with M-Ras-1, M-Ras-2 or M-Ras-3 three mutants expected to be constitutively activated, the cells did not die in the absence of IL-3 but continued to proliferate (bottom panel). In summary, this experiment indicates that activation of M-Ras protects against apoptosis induced by IL-3 withdrawal in BaF3 cells and induces cytokine-independent proliferation in the same cells. This effect on survival and proliferation suggests a potential oncogenic activity of activating M-Ras mutations.

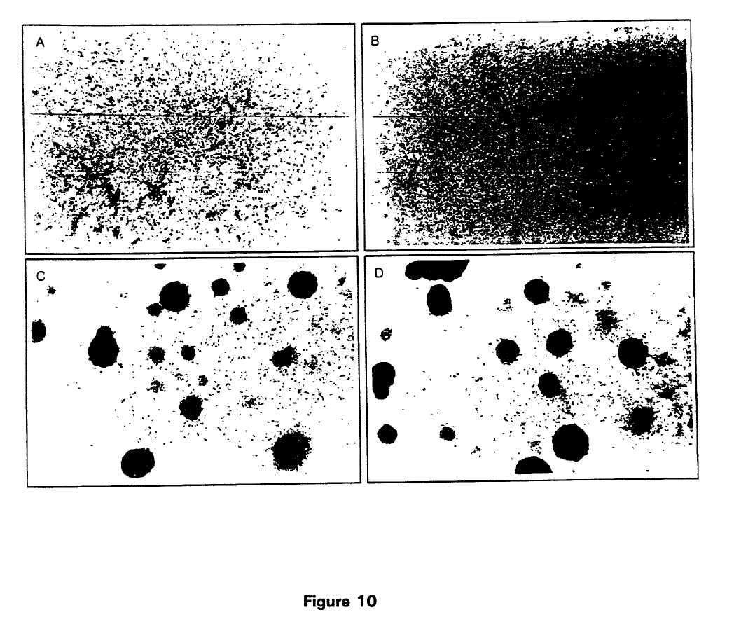

The observation that activated M-Ras induced survival and cytokine-independent proliferation of IL-3 deprived BaF3 cells is suggestive of the oncogenic activity of these M-Ras mutations. To confirm the transforming potential of activated M-Ras, applicants used the classical foci formation assay with NIH3T3 fibroblasts. Cells were transfected with either wild-type M-Ras, constitutively active M-Ras-i or with activated H-Ras as a positive control. Foci formation was not observed in cells transfected with vector alone (FIG. 10A) or wild-type M-Ras (FIG. 10B) whereas transfection with M-Ras-1 (FIG. 10C) or activated H-Ras (FIG. 10D) resulted in foci formation overgrowing a stationary monolayer of NIH3T3 cells. Aberrant M-Ras activation can therefore cause malignant transformation of NIH3T3 cells suggesting that deregulation of M-Ras function may also contribute to spontaneous malignancies, particularly in IL-9 transgenic mice. In these mice, constitutive IL-9 overexpression indeed results in a high susceptibility to the development of T cell lymphomas upon exposure to low doses of chemical mutagen, which was reported to induce Ras mutations (Renauld et al., 1994).

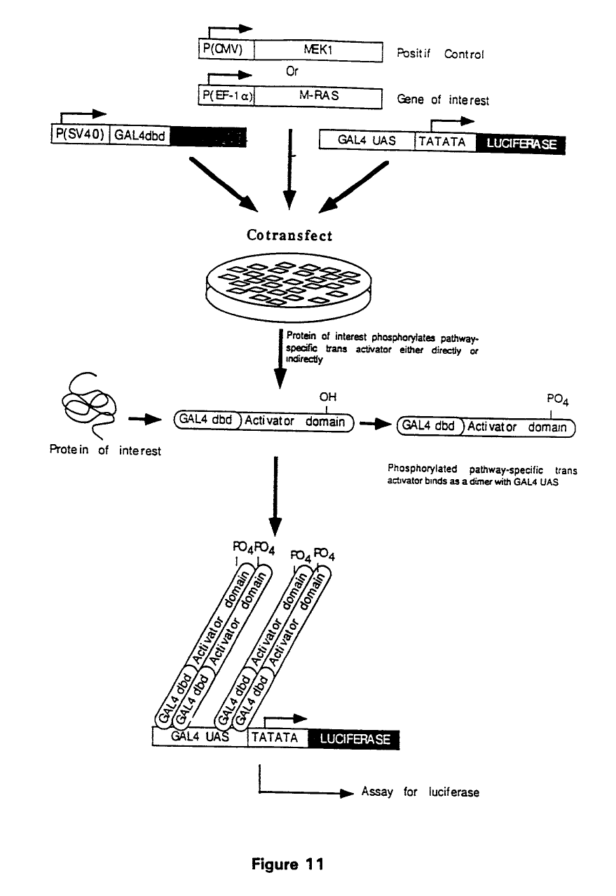

In order to characterize the pathway stimulated by activated M-Ras, applicants tested the ability of activated mutants to up-regulate the mitogen-activated protein kinase (MAPK) pathway or the c-Jun N-terminal kinase (JNK) pathway using an in vitro signal transduction pathway reporting system (FIG. 11). This luciferase-based assay allows for indirect measurement of Elk (target of the MAPK pathway) or Jun (target of the JNK pathway) protein activation. Activated MEK1, which activates Elk, served as a positive control for the MAPK pathway whereas activated MEKK was used as a positive control for the JNK pathway because it activates Jun.

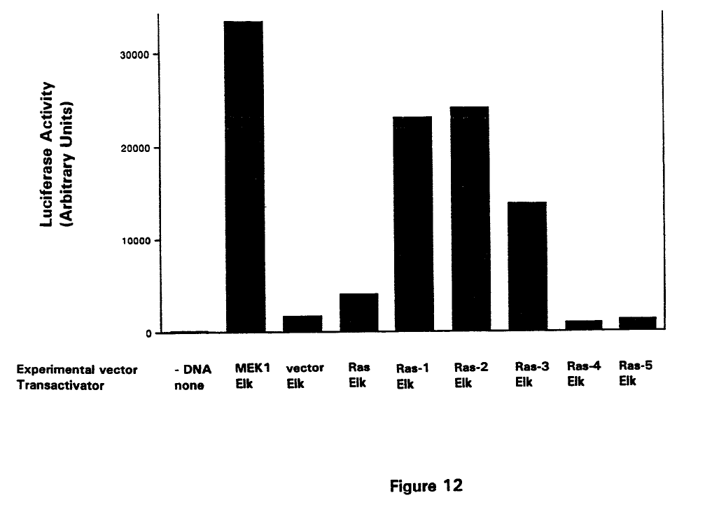

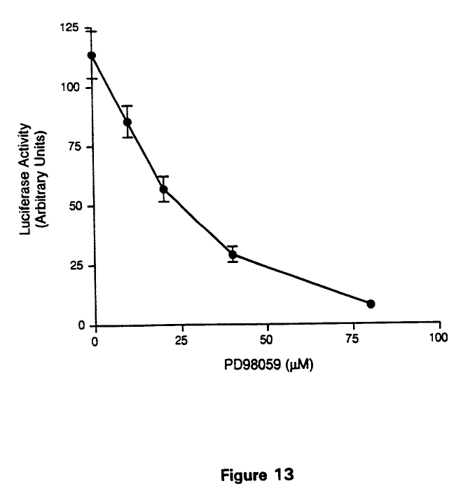

The results of this experiment are shown in FIG. 12 for the MAPK pathway. As expected, activated MEK1 expression resulted in Elk protein (target of MAPK pathway) activation. In addition, M-Ras-1, M-Ras-2 and M-Ras-3 mutants activated Elk, indicating M-Ras induced activation of the MAPK pathway. The induction of Elk by these mutants was blocked by the PD98059 compound (FIG. 13), which is known to be an inhibitor of the MEK1 kinase, further demonstrating activation of the MAPK pathway and indicating that M-Ras is involved upstream from this kinase. The same results were obtained in the P815 murine mastocytoma cells, indicating that activation of this pathway is not cell type- or species-specific. When the same experiment was performed for the JNK pathway, activated M-Ras mutants did not show any activation whereas activated MEKK did, indicating that M-Ras is not involved in this pathway.

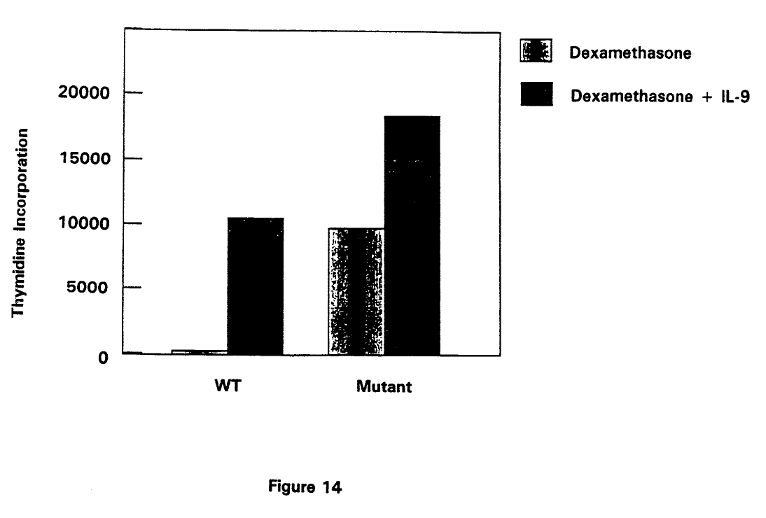

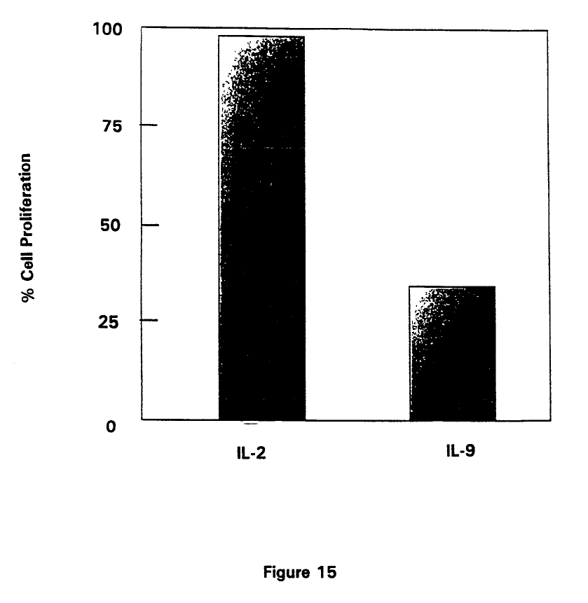

The role of M-Ras in IL-9 signaling is suggested by the finding that expression of a constitutively active mutant M-Ras in BW5147 cells, which undergo apoptosis upon treatment with dexamethasone that can be rescued by IL-9, abrogates the requirement for IL-9 for anti-apoptotic activity (FIG. 14). The selectivity of this pathway is demonstrated by the ability to block constitutively activated mutants of this gene using MAPK inhibitors which have also been shown to block IL-9 induced proliferation of a T-cell line while no effect was observed when these same cells were grown in the presence of another cytokine (FIGS. 15, 16 and 17).

Constitutively activated mutants of this gene were also found to mediate the cytokine-independent survival and proliferation in the murine BaF3 cell line and to activate the MAPK signal transduction pathway in the HEK293 cells and murine P815 mastocytoma. Based on observations made with other Ras genes, for which the very same activating mutations were found in many types of tumors, it is likely that M-Ras mutations are also involved in human cancer.

Further evidence defining the role of M-Ras in the pathogenesis of atopic allergy, bronchial hyperresponsiveness, asthma and related disorders derives directly from the applicants' observation that IL-9 selectively induces M-Ras. Thus, the pleiotropic role for IL-9 which is critical to a number of antigen induced responses is, in part, dependent on the regulation of M-Ras which plays a role in the physiology of a number of cells critical to atopic allergy. When the functions of IL-9 are down-regulated by antibody pretreatment prior to aerosol challenge with antigen, the animals can be completely protected from the antigen induced responses. These responses include: bronchial hyperresponsiveness, eosinophilia and elevated cell counts in bronchial lavage, histologic changes in lung associated with inflammation and elevated total serum IgE. Thus, the treatment of such responses by down-regulating M-Ras, which are critical to the pathogenesis of atopic allergy and which characterize the allergic inflammation associated with atopic allergy, are within the scope of this invention.

Applicants also teach the down-regulation of M-Ras by administering antagonists of M-Ras. The skilled artisan will readily recognize that all molecules containing the requisite three-dimensional structural conformation critical for activation or ligand binding to M-Ras are within the scope of this invention. The structure of M-Ras has been examined and analyzed in great detail and amino acid residues of M-Ras critical for activation or binding to ligand have been identified. These sites include but are not limited to residues 20-27 comprising the ATP/GTP binding site, residues 193-195 comprising a RGD sequence likely involved in adhesion to cell structural proteins and residues 205-208 which represent the site for famesyl transferase or geranylgeranyl transferase activation. Farnesyl transferase and geranylgeranyl transferase inhibitors are well known in the art and examples of such inhibitors have been previously described (Qian et al., 1997). The use of such inhibitors for down-regulation of M-Ras are within the claimed invention. Such famesyl transferase inhibitors include but are not limited to manumycin A and lovastatin. Peptides derived from the above sites are also included in the invention, particularly peptides which modulate the interaction of a specific ligand with one of the above identified sites.

The demonstration of an IL-9 sequence associated with an asthma-like phenotype and one associated with the absence of an asthma-like phenotype, indicates that the inflammatory response to antigen in the lung is IL-9 dependent and therefore, down-regulating M-Ras, which is selectively induced downstream in the IL-9 pathway, will protect against the antigen induced response. Furthermore, applicant also provides methods of diagnosing susceptibility to atopic allergy and related disorders and for treating these disorders based on the relationship between IL-9, its receptor and M-Ras.

The present invention also includes antagonists of M-Ras that block activation of this protein (antagonists may also be referred to as inhibitors). Antagonists are compounds that are themselves devoid of pharmacological activity but cause effects by preventing the action of an agonist. To identify an antagonist of the invention, one may test for competitive binding with natural ligands (or substrates) of M-Ras. Assays of antagonistic binding and activity can be derived from monitoring M-Ras functions for down-regulation as described herein and in the cited literature. One may test for binding to M-Ras to identify allosteric ligands or inverse agonists of the invention. The binding of antagonist may involve all known types of interactions including ionic forces, hydrogen bonding, hydrophobic interactions, van der Waals forces and covalent bonds. In many cases, bonds of multiple types are important in the interaction of an antagonist with a molecule like M-Ras.

In a further embodiment, these compounds may be analogues of M-Ras or its ligands. M-Ras analogues may be produced by point mutations in the isolated DNA sequence for the gene, nucleotide substitutions and/or deletions which can be created by methods that are all well described in the art (Simoncsits et al., 1994). This invention also includes spliced variants of M-Ras including isolated nucleic acid sequences of M-Ras, which contain deletions of one or more of its exons. The term “spliced variants” as used herein denotes a purified and isolated DNA molecule encoding human M-Ras comprising at least one axon. In addition, these exons may contain various point mutations.

Structure-activity relationships may be used to modify the antagonists of the invention. For example, the techniques of X-ray crystallography and NMR may be used to make modifications of the invention. For example, one can create a three dimensional structure of human M-Ras that can be used as a template for building structural models of deletion mutants using molecular graphics. These models can then be used to identify and construct a ligand for M-Ras with affinity comparable to the natural ligand, but with lower M-Ras activity when compared to the natural ligands. What is meant by lower biologic activity is 2 to 100,000 fold less M-Ras activity than produced by natural ligands, preferably 100 to 1,000 fold less M-Ras activity than produced by natural ligands. In still another embodiment, these compounds may also be used as dynamic probes for M-Ras structure and to develop M-Ras antagonists using cell lines or other suitable means of assaying M-Ras activity.

In addition, this invention also provides compounds and methods of screening for compounds that prevent the synthesis or reduce the biologic stability of M-Ras. Biologic stability is a measure of the time between the synthesis of the molecule and its degradation. For example, the stability of a protein, peptide or peptide mimetic (Kauvar, 1996) therapeutic may be shortened by altering its sequence to make it more susceptible to enzymatic degradation.

The present invention also includes methods of screening for compounds which activate, or act as agonists, of mRas or mRas expression. Such compounds may be useful in the modulation of pathological conditions, for instance, conditions associated with IL-9 receptor deficiencies. Such compounds may also be useful in modulating other deficiencies in IL-9, IL-9 induced conditions or 11-9 induced physiological states.

One diagnostic embodiment involves the recognition of variations in the DNA sequence of M-Ras. One method involves the introduction of a nucleic acid molecule (also known as a probe) having a sequence complementary to the M-Ras of the invention under sufficient hybridizing conditions, as would be understood by those in the art. In one embodiment, the sequence will bind specifically to one allele of M-Ras or a fragment thereof and in another embodiment will bind to multiple alleles. Another method of recognizing DNA sequence variation associated with these disorders is direct DNA sequence analysis by multiple methods well known in the art (Ott, 1991). Another embodiment involves the detection of DNA sequence variation in the M-Ras gene associated with these disorders (Schwengel et al., 1993; Sheffield et al., 1993; Orita et al., 1989; Sarkar et al., 1992; Cotton, 1989). These include the polymerase chain reaction, restriction fragment length polymorphism analysis and single stranded conformational analysis.

In another diagnostic embodiment, susceptibility to asthma-related disorders and certain lymphomas and leukemias associated with elevated levels of M-Ras polypeptide in a human subject can be measured by the steps of: (a) measuring the level of M-Ras polypeptide in a biological sample from said human subject; and (b) comparing the level of M-Ras polypeptide present in normal subjects, wherein an increase in the level of M-Ras polypeptide as compared to normal levels indicates a predisposition to asthma-related disorders and certain lymphomas or leukemias. Such lymphomas and leukemias include adult diffuse aggressive lymphoma, peripheral T-cell lymphoma, thymic lymphoma, Hodgkin lymphoma, lymphoblastic lymphomas, chronic lymphocytic leukemia, large granular lymphocyte leukemia, myeloid leukemia, HTLV-induced T cell leukemia, adult T-cell leukemia and acute lymphocytic leukemia.

In another diagnostic embodiment, a therapeutic treatment of asthma-related disorders or certain lymphomas or leukemias associated with elevated levels of M-Ras polypeptide in a human subject may be monitored by measuring the levels of M-Ras polypeptide in a series of biologic samples obtained at different time points from said subject undergoing therapeutic treatment wherein a significant decrease in said levels of M-Ras polypeptide indicates a successful therapeutic treatment.

Diagnostic probes useful in such assays of the invention include antibodies to M-Ras. The antibodies to M-Ras may be either monoclonal or polyclonal, produced using standard techniques well known in the art (See Harlow & Lane's Antibodies: A Laboratory Manual, Cold Spring Harbor Laboratory Press, 1988). They can be used to detect M-Ras by binding to the protein and subsequent detection of the antibody-protein complex by ELISA, Western blot or the like. The M-Ras used to elicit these antibodies can be any of the M-Ras variants discussed above. Antibodies are also produced from peptide sequences of M-Ras using standard techniques in the art (See Protocols in Immunology, John Wiley & Sons, 1994). The peptide sequence from M-Ras that can be used to produce blocking antisera has been identified as CKKKTKWRGDRATGTHKLQ (residues 187-204) (SEQ ID NO:5). Fragments of the monoclonals or the polyclonal antisera which contain the immunologically significant portion can also be prepared. Use of immunologically reactive fragments, such as the Fab, Fab′, of F(ab′)₂ fragments is often preferable, especially in a therapeutic context, as these fragments are generally less immunogenic than the whole immunoglobulin.

Assays to detect or measure M-Ras polypeptide in a biological sample with an antibody probe may be based on any available format. For instance, in immunoassays where M-Ras polypeptides are the analyte, the test sample, typically a biological sample, is incubated with anti-M-Ras antibodies under conditions that allow the formation of antigen-antibody complexes. Various formats can be employed, such as “sandwich” assay where antibody bound to a solid support is incubated with the test sample; washed, incubated with a second, labeled antibody to the analyte; and the support is washed again. Analyte is detected by determining if the second antibody is bound to the support. In a competitive format, which can be either heterogeneous or homogeneous, a test sample is usually incubated with an antibody and a labeled competing antigen, either sequentially or simultaneously. These and other formats are well known in the art.

A further embodiment of the invention relates to antisense or gene therapy. It is now known in the art that altered DNA molecules can be tailored to provide a specific selected effect, when provided as antisense or gene therapy. The native DNA segment coding for M-Ras has, as do all other mammalian DNA strands, two strands; a sense strand and an antisense strand held together by hydrogen bonds. The mRNA coding for M-Ras has a nucleotide sequence identical to the sense strand, with the expected substitution of thymidine by uridine. Thus, based upon the knowledge of the M-Ras sequence, synthetic oligonucleotides can be synthesized. These oligonucleotides can bind to the DNA and RNA coding for M-Ras. The active fragments of the invention, which are complementary to mRNA and the coding strand of DNA, are usually at least about 15 nucleotides, more usually at least 20 nucleotides, preferably 30 nucleotides and more preferably may be 50 nucleotides or more. There is no upper limit, other than a practical limit, on the maximal size of such a nucleic acid molecule in that the nucleic acid molecule can include a portion of a gene, an entire gene, or multiple genes, or portions thereof. The binding strength between the sense and antisense strands is dependent upon the total hydrogen bonds. Therefore, based upon the total number of bases in the mRNA, the optimal length of the oligonucleotide sequence may be easily calculated by the skilled artisan. The sequence may be complementary to any portion of the sequence of the mRNA. For example, it may be proximal to the 5′-terminus or capping site or downstream from the capping site, between the capping site and the initiation codon and may cover all or only a portion of the non-coding region or the coding region. The particular site(s) to which the antisense sequence binds will vary depending upon the degree of inhibition desired, the uniqueness of the sequence, the stability of the antisense sequence, etc.

In the practice of the invention, expression of M-Ras is down-regulated by administering an effective amount of synthetic antisense oligonucleotide sequences described above. The oligonucleotide compounds of the invention bind to the mRNA coding for human M-Ras thereby inhibiting expression (translation) of these proteins. The isolated DNA sequences containing various mutations such as point mutations, insertions, deletions or spliced mutations of M-Ras are useful in gene therapy as well.

Antisense oligonucleotides can also be used as tools in vitro to determine the biological function of genes and proteins. Oligonucleotide phosphorothioates (PS-oligos) have also shown great therapeutic potential as antisense-mediated inhibitors of gene expression (Stein et al., 1993 and references therein). Various methods have been developed for the synthesis of antisense oligonucleotides. See Agrawal et al., Methods of Molecular Biology: Protocols for Oligonucleotides and Analogs, Humana Press, 1993 and Eckstein et al., Oliconucleotides and Analogues: A Practical Approach, Oxford University Press, 1991).

The present invention also provides transgenic animals that over-express M-Ras or express M-Ras at a level much lower than that of a wild-type organism. A “wild type” organism is one that is the most frequently observed phenotype for M-Ras expression, usually arbitrarily designated as a “normal” individual.

Transgenic animals are genetically modified animals into which cloned genetic material has been transferred. The cloned genetic material is often referred to as a transgene. The transgene may consist of nucleic acid sequences derived from the genome of the same species or of a different species, including non-animal species, than the species of the target animal.

The development of transgenic technology allows investigators to create mammals of virtually any genotype and to assess the consequences of introducing specific foreign nucleic acid sequences on the physiological and morphological characteristics of the transformed animals. The availability of transgenic animals permits cellular processes to be influenced and examined in a systematic and specific manner not achievable with most other test systems. For example, the development of transgenic animals provides biological and medical scientists with models that are useful in the study of disease. Such animals are also useful for the testing and development of new pharmaceutically active substances. Gene therapy can be used to ameliorate or cure the symptoms of genetically-based diseases.

Transgenic animals can be produced by a variety of different methods including transfection, electroporation, microinjection, biolistics (also called gene particle acceleration or microprojectile bombardment), gene targeting in embryonic stem cells and recombinant viral and retro viral infection (See U.S. Pat. No. 4,736,866; U.S. Pat. No. 5,602,307; Mullins et al., 1993; Brenin et al., 1997; Tuan, Recombinant Gene Expression Protocols. Methods in Molecular Biology, Humana Press, 1997.

The term “knock-out” generally refers to mutant organisms which contain a null allele of a specific gene. The term “knock-in” generally refers to mutant organisms into which a gene has been inserted through homologous recombination. The knock-in gene may be a mutant form of a gene which replaces the endogenous, wild-type gene. Mice which are knock-in or knock-out mice as regards the M-Ras gene are encompassed by the disclosure of this invention.

A number of recombinant rodents have been produced, including those which express an activated oncogene sequence (U.S. Pat. No. 4,736,866); express simian SV 40 T-antigen (U.S. Pat. No. 5,728,915); lack the expression of interferon regulatory factor-1 (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); and also possess an bovine growth hormone gene (Clutter et al., 1996).

While rodents, especially mice and rats, remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (See Kim et al., 1997; Houdebine, 1995; Petters, 1994; Schnieke et al., 1997; and Amoah et al., 1997).

The method of introduction of nucleic acid fragments into recombination competent mammalian cells can be by any method which favors co-transformation of multiple nucleic acid molecules. Detailed procedures for producing transgenic animals are readily available to one skilled in the art, including the recitations in U.S. Pat. No. 5,489,743 and U.S. Pat. No. 5,602,307.

In addition to the direct inhibition of the M-Ras gene, this invention also encompasses methods of inhibition of intracellular signaling by M-Ras. It is known in the art that highly exergonic phosphoryl-transfer reactions are catalyzed by various enzymes known as kinases. In other words, a kinase transfers phosphoryl groups between ATP and a metabolite. Included within the scope of this invention are specific inhibitors of protein kinases. Thus, inhibitors of these kinases are useful in the down-regulation of M-Ras and are useful in the treatment of atopic allergies and asthma.

In still another aspect of the invention, surprisingly, aminosterol compounds were found to be useful in the inhibition of M-Ras induction by mitogen stimulation. Aminosterol compounds which are useful in this invention are described in U.S. patent application Ser. No. 08/290,826 and its related application Ser. Nos. 08/416,883 and 08/478,763 as well as in Ser. No. 08/483,059 and its related application Ser. Nos. 08/483,057, 08/479,455, 08/479,457, 08/475,572, 08/476,855, 08/474,799 and 08/487,443, which are specifically incorporated herein by reference. The ability of an aminosterol compound to block M-Ras induction could be determined by any one of numerous assays previously described in the art which screen for signaling partners of Ras proteins (Kimmelman et al., 1997; Vojtek et al., 1993).

In addition, the invention includes pharmaceutical compositions comprising the compounus of the invention together with a pharmaceutically acceptable carrier. Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Reminaton's Pharmaceutical Sciences, Mack Publishing Company, 1995, specifically incorporated herein by reference.

The compounds used in the method of treatment of this invention may be administered systemically or topically, depending on such considerations as the condition to be treated, need for site-specific treatment, quantity of drug to be administered and similar considerations.

Topical administration may be used. Any common topical formulation such as a solution, suspension, gel, ointment or salve and the like may be employed. Preparation of such topical formulations as are well described in the art of pharmaceutical formulations as exemplified, for example, by Remington's Pharmaceutical Sciences. For topical application, these compounds could also be administered as a powder or spray, particularly in aerosol form. The active ingredient may be administered in pharmaceutical compositions adapted for systemic administration. As is known, if a drug is to be administered systemically, it may be confected as a powder, pill, tablets or the like or as a syrup or elixir for oral administration. For intravenous, intraperitoneal or intra-lesional administration, the compound will be prepared as a solution or suspension capable of being administered by injection. In certain cases, it may be useful to formulate these compounds in suppository form or as an extended release formulation for deposit under the skin or intramuscular injection. In a preferred embodiment, the compounds of this invention may be administered by inhalation. For inhalation therapy the compound may be in a solution useful for administration by metered dose inhalers or in a form suitable for a dry powder inhaler.

An effective amount is that amount which will down-regulate M-Ras. A given effective amount will vary from condition to condition and in certain instances may vary with the severity of the condition being treated and the patient's susceptibility to treatment. Accordingly, a given effective amount will be best determined at the time and place through routine experimentation. However, it is anticipated that in the treatment of atopic allergy and asthma-related disorders in accordance with the present invention, a formulation containing between 0.001 and 5 percent by weight, preferably about 0.01 to 1%, will usually constitute a therapeutically effective amount. When administered systemically, an amount between 0.01 and 100 mg per kg body weight per day, but preferably about 0.1 to 10 mg/kg, will effect a therapeutic result in most instances.

The practice of the present invention will employ the conventional terms and techniques of molecular biology, pharmacology, immunology and biochemistry that are within the ordinary skill of those in the art. For example, see Sambrook et al., Molecular Cloning: A Laboratory Manual, 2nd edition, Cold Spring Harbor Laboratory Press, 1985.

Other embodiments of the invention will be apparent to those skilled in the art from consideration of the specification and practice of the invention disclosed. It is intended that the specifications and examples be considered exemplary only with a true scope of the invention being indicated by the claims. Having provided this background information, applicant now describes preferred aspects of the invention M-Ras.

### EXAMPLE 1

### cDNA Difference Analysis of IL-9 Induced Genes

A murine T lymphocyte cell clone TS2 was used to isolate IL-9 induced genes. TS2 is a T helper cell line derived from primary culture of murine lymphocytes as previously described (Uyttenhove et al., 1988; Louahed et al., 1995). This cell line has been shown to proliferate in response to IL-2, IL-4 or IL-9 cytokines in culture. In order to identify IL-9 specifically induced genes cDNA difference analysis was performed on mRNA from cells cultured in the presence of IL-2 or IL-9.

Cell Culture and Cytokines.

TS2 cells were grown in DMEM medium supplemented with 10% fetal calf serum, 50 μM 2-mercaptoethanol, 0.55 mM L-arginine, 0.24 mM L-asparagine and 1.25 mM L-glutamine. This factor dependent cell line was able to grow in the presence of either IL-2, IL-4 or IL-9 without antigen or feeder cells.

cDNA Synthesis.

Total RNA was prepared from TS2 cells stimulated with IL-2 (200 U/ml) or IL-9 (200 U/ml) for 48 hours by the guanidine isothiocyanate method (Chomczynski et al., 1987). Polyadenylated RNA was purified from total RNA with oligo(dT) cellulose columns. Double stranded cDNA was prepared by reverse transcription using Superscript II reverse transcriptase and an oligo(dT) primer as suggested by the manufacturer (Gibco-BRL). cDNA was then prepared for cDNA difference analysis by phenol-chloroform extraction and ethanol precipitation. Products were resuspended in nuclease free water and analyzed on agarose to determine quality of products as described below.

cDNA Difference Analysis Protocol.

Differential cDNA analysis of TS2 cells treated with IL-2 or IL-9 was carried out as previously described (Hubank et al., 1994), based on the genomic cDNA difference analysis procedure of Lisitsyn et al., 1993.

Oligo(dT) primers were used to generate cDNA from cytoplasmic polyadenylated mRNA isolated from TS2 cells. cDNA was digested with Dpnil followed by two extractions with phenoi-chloroform-isoamyl alcohol and one with chloroform-isoamyl alcohol. A glycogen carrier was added followed by precipitation with 100% ethanol. The pellet was washed with 70% ethanol, dried and resuspended in TE buffer.

For ligation of adaptors, digested cDNA was combined with desalted R-Bgl-24 (5′-AGCACTCTCCAGCCTCTCACCGCA-3′) (SEQ ID NO:6), R-Bgl-12 (5′-GATCTGCGGTGA-3′) (SEQ ID NO:7) oligos (2:1 ratio), 10× ligase buffer and water. Oligos were annealed to digested cDNA at 50° C. for 1 minute then cooled to 10° C. for 1 hour followed by addition of T4 DNA ligase and overnight incubation at 16° C. Ligation reactions were then diluted with TE buffer.

Generation of Representations.

Diluted ligation reaction was combined with 5× PCR buffer, dNTP nucleotide mix, water and R-Bgl-24 primer. The reaction was heated to 72° C. for 3 minutes to remove the 12-mer followed by addition of Taq DNA polymerase. The reactions were then incubated for 5 minutes at 72° C. to fill in the ends followed by a PCR cycling protocol of 20 cycles (1 minute at 95° C., 3 minutes at 72° C.). A final extension step (10 minutes at 72° C.) was included at the end of the cycling protocol.

PCR products were extracted twice with phenol-chloroform-isoamyl alcohol, once with chloroform-isoamyl alcohol and precipitated with isopropanol. Pellets were washed with 70% ethanol and resuspsended in TE buffer. Each representation was then digested with Dpnll followed by one extraction with phenol-chloroform and another with chloroform. Digested representations were precipitated with isopropanol and washed with 70% ethanol and resuspended in TE buffer. This DNA was designated the cut DRIVER.

Preparation of the TESTER.

Digested representation was diluted with TE buffer and combined with 10× loading buffer loaded onto a 1.2% TAE prep gel and electrophoresed until the bromphenol blue had migrated approximately 2 cm. The amplicon-containing portion of the gel was excised, separating it from the digested linkers. This DNA was purified from the gel slice and resuspended in TE buffer and was designated the TESTER.

Ligation of TESTER to the J-oligos.

TESTER was combined with 10× ligase buffer, water, desalted J-Bgl-24 (5′-ACCGACGTCGACTATCCATGMCA-3) (SEQ ID NO:8) and J-Bgl-12 (5′-GATCTGTTCATG-3′) (SEQ ID NO:9) oligos (2:1 ratio) then annealed to TESTER at 50° C. for 1 minute then cooled to 10° C. for 1 hour followed by addition of T4 DNA ligase and overnight incubation at 16° C. Ligation reactions were then diluted with TE buffer.

Subtractive Hybridization.

The digested DRIVER representation and J-ligated TESTER representation were combined followed by extraction with phenol-chloroform. DNA was precipitated with 100% ethanol and washed twice with 70% ethanol, dried and resuspended in TE buffer. Reaction was overlaid with mineral oil and denatured for 5 minutes at 98° C., cooled to 67° C., followed by addition of 5 M NaCI and incubation for 20 hours to allow for complete hybridization.

Generation of First Difference Product.

Mineral oil was removed and DNA was diluted with TE buffer and 5 μg/μl yeast RNA. For each subtraction setup, diluted hybridization mix was combined with 5× PCR buffer, dNTP nucleotide mix and water. The reactions were incubated at 72° C. for 3 minutes to remove the 12-mer, Taq DNA polymerase added, incubated another 5 minutes at 72° C., J-Bgl-24 primer added followed by a PCR cycling protocol of 10 cycles (1 minute at 95° C., 3 minutes at 70° C.). A final extension step (10 minutes at 72° C.) was included at the end of the cycling protocol. Reactions were extracted with phenol-chloroform-isoamyl alcohol and once with chloroform-isoamyl alcohol. A glycogen carrier was added followed by precipitation with 100% ethanol. The pellet was washed with 70% ethanol and resuspended in 0.2× TE buffer.

PCR products were then digested with mung bean nuclease for 35 minutes at 30° C. and the reaction stopped by incubation for 5 minutes at 98° C. in the presence of 50 mM Tris-HCI. Mung bean nuclease-treated DNA was combined with 5× PCR buffer, dNTP nucleotide mix, water and J-Bgl-24 oligo. Reactions were incubated 1 minute at 95° C., cooled to 80° C. and Taq DNA polymerase added followed by a PCR cycling protocol consisting of 18 cycles (1 minute at 95° C., 3 minutes at 70° C.). A final extension step (10 minutes at 72° C.) was included at the end of the cycling protocol. PCR products were extracted twice with phenol-chloroform-isoamyl alcohol and once with chloroform-isoamyl alcohol. DNA was precipitated with isopropanol, washed with 70% ethanol and resuspended in TE buffer. This reaction product was designated the first difference product (DPI).

Change of Adaptors on a Difference Product.

DP1 was digested with Dpnll, extracted twice with phenol-chloroform-isoamyl alcohol and precipitated with 100% ethanol. The pellet was washed with 70% ethanol and resuspended in TE buffer. Template DNA was combined with digested DP1, 10× ligase buffer, water, N-Bgl-24 (5′-AGGCMCTGTGCTATCCGAGGGAA-3′) (SEQ ID NO:10) and N-Bgl-12 (5′-GATCTTCCCTCG-3′) (SEQ ID NO:11) oligos (2:1 ratio) then annealed at 50° C. for 1 minute then cooled to 10° C. for 1 hour followed by addition of T4 DNA ligase and overnight incubation at 16° C.

Generation of second (DP2) and third difference product (DP3). For DP2, N-ligated DP1 was mixed with DRIVER and subtraction and amplification steps (1:800 TESTER:DRIVER ratio) were repeated as described above (J oligos were used for ligation step). For DP3, J-ligated DP2 was diluted with TE buffer containing 5 μg/μl yeast RNA. J-ligated DP2 was hybridized with DRIVER and subtraction and amplification steps (1:400,000 TESTER:DRIVER ratio) were repeated as described above to generate DP3, performing the final amplification protocol for 22 cycles.

Cloning of DP3 was achieved by digesting DP3 with Dpnil, isolation on TAE prep gel as described above, purification from the excised band and cloning into pTZ19R vector. Difference products were initially characterized by: conformation of genuine difference product by probing against a blot of the original amplicons, conformation by sequencing or Northern blots to determine whether difference products originated from more than one transcript, ascertaining the frequency of cloning by probing a plasmid blot of cloned DP3 minipreps and sequencing genuine differences for potential identification via BLASTmail.

### EXAMPLE 2

### Identification of the Murine M-Ras Induced by IL-9

One of several cDNA identified from the difference analysis was found to be a novel cDNA. A full-length cDNA was cloned from a murine cDNA library using the fragment isolated as a probe. A TS2 cDNA library was prepared by conventional methods in the pSVK3 plasmid library as previously described (Louahed et al., 1995). A 1119 bp cDNA was isolated which contained an open reading frame encoding for a protein of 208 amino acids which is M-Ras. FIG. 1 shows the nucleotide and amino acid sequence of the M-Ras cDNA. A nucleotide BLAST (Altschul et al., 1990) database search of the full length cDNA revealed it to be similar to several Ras proteins. FIG. 2 shows an alignment of M-Ras to H-Ras and R-Ras proteins. Motif analysis of the encoded polypeptide shows several features such as a nucleotide binding domain in the N-terminus and a CAAX motif at the C-terminus. These are hallmark features of other Ras protein members and suggests that M-Ras is an IL-9 inducible gene involved in signal transduction.

### EXAMPLE 3

### M-Ras is Induced in Vitro by IL-9 in Murine Cells.

To confirm that M-Ras is induced by IL-9, murine T-helper cell line TS2 was cultured with 200 U/ml murine IL-9 or murine IL-2 (R&D Systems). Cells were counted and total RNA was extracted from equivalent number of cells as described in Example 1. Total cellular RNA was fractionated by electrophoresis, transferred to Hybond-C nitrocellulose membrane (Amersham) and probed. The M-Ras probe was a 1.2 kb cDNA containing the complete coding sequence for M-Ras which was labeled using the Multiprime DNA labeling kit (Amersham). Following autoradiography, all blots were reprobed with a chicken β-actin probe to control for even loading of RNA. For RT-PCR analysis, cDNA was generated using random hexamers (Pharmacia) and Superscript II (Gibco-BRL) as suggested by the manufacturer. Message was analyzed by PCR as described in Example 1. Primers used to generate murine M-Ras message were: sense 5′-CCAGACTGGCACAGTTCC-3′ (SEQ ID NO:12) (codons 3-8) and antisense 5′-TGCTGTAGAAGCCGAAGCC-3′ (SEQ ID NO:13) (codons 86-92) which produce a gene product of 267 bp. β-actin was assayed as an internal control to measure for cDNA integrity using primers previously described (Nicolaides et al., 1991). Amplification conditions used were 95° C. for 30 seconds, 58° C. for 1.5 minutes and 72° C. for 1.5 minutes for 35 cycles.

The results of the in vitro experiments showed that M-Ras is specifically expressed in the cytokine dependent murine cell line TS2 when cultured in the presence of IL-9. The specific induction by IL-9 is demonstrated by the fact that the gene is expressed in the presence of IL-9 but not IL-2 as determined by cDNA difference analysis. The induction of M-Ras by IL-9 in TS2 cells was found to appear at approximately 6 hours and express maximally at 24 hours after exposure to IL-9 (FIG. 3). This data demonstrates a direct effect of IL-9 on M-Ras expression, where IL-9 responsive cells produce M-Ras for intracellular signaling.

### EXAMPLE 4

### In vivo Expression of Murine M-Ras.

In vivo M-Ras gene expression was assessed in different murine tissues containing either an IL-9 transgene (Tg5) or the parental strain (FVB) of the IL-9 gene. The IL-9 transgenic mice were generated as described (Renauld et al., 1994). Mice were euthanized and various organs were aseptically harvested and prepared for total RNA extraction using the guanidine isothiocyanate method described above. RNA was processed, reverse transcribed and PCR amplified using primers for M-Ras and Mactin as described in Example 3. Products were electrophoresed on agarose gels and stained with ethidium bromide.

RT-PCR analysis of RNA derived from the tissues of the IL-9 transgenic (Tg5) and the parental strain (FVB) revealed that both strains expressed M-Ras in the kidney, lung and brain (FIG. 4). This data demonstrates that the IL-9 inducible gene M-Ras is expressed in several tissues in mice including the lung. This data also suggests that M-Ras may play a role in the physiology of these organs.

### EXAMPLE 5

### The Cloning of the Human M-Ras Homolog

A human homologue cDNA was identified by RT-PCR using oligonucleotides based on the murine M-Ras sequence. A derived human specific probe was then used to screen a human testis cDNA library (cloned in pcDNAamp), from which several cDNA clones were isolated. Briefly, 10,000 recombinant bacteria were plated onto nitrocellulose membranes and plates were incubated overnight at 37° C. Duplicate filters were prepared for each membrane and one of them was hybridized with a human M-Ras probe radiolabeled with α ³²P-dCTP using the Rediprime random prime labeling kit (Amersham). Filters were hybridized in 3.5× SSC, 1× Denhardt, 25 mM NaHPO₄, 2 mM EDTA, 0.5% SDS and 200 μg/ml denatured salmon sperm DNA. Hybridizations were carried out for 16 hours at 65° C. with a final probe concentration of 2×10⁶ cpm/ml, followed by three washes in 0.2× SSC and 0.1% SDS. Membranes were then exposed to film overnight and colonies that exhibited a strong signal were selected. Double stranded plasmid DNA was prepared and sequenced with the Thermo-sequenase sequencing kit (Amersham). The largest of these cDNA clones contained an 1081 base pair insert which included an open reading frame encoding a protein of 208 amino acids with a calculated molecular weight of 23,831 Daltons. There is a 89% nucleotide sequence identity between the human and mouse coding regions and a 97% amino acid sequence identity between the corresponding proteins.

### EXAMPLE 6

### Distribution of M-Ras in Human Tissues

To determine in which tissues M-Ras was expressed, RT-PCR was performed using RNA extracted from various tissues as described in Example 1. cDNA was then PCR amplified using the oligodeoxynucleotide primers, sense 5′-GAATTCAGCGCCATGCGC-3′ (SEQ ID NO:14) (centered at nucleotide 327) and antisense 5′-CCTCACMGATCACACATTG-3′ (SEQ ID NO:15) (centered atnucleotide 721)which results in a productof413 bp. β-actin was used as a control to monitor for cDNA integrity as described in Example 3. PCR amplifications were carried out at 95° C. for 30 seconds, 58° C. for 1.5 minutes and 72° C. for 1.5 minutes for 35 cycles. Reactions were electrophoresed in 2.5% agarose gels and stained with ethidium bromide. M-Ras expression was standardized using Mactin as a control for input material. As shown in FIG. 6, M-Ras expression was detected in most tissues except for the colon and liver. It was found to be most abundant in the adrenal gland, breast, lung and brain, of which the last two were found expressed in murine tissue. This data further suggests a biological role for the IL-9 induced M-Ras in these tissues.

### EXAMPLE 7

### IL-9 Inducibility of Human M-Ras

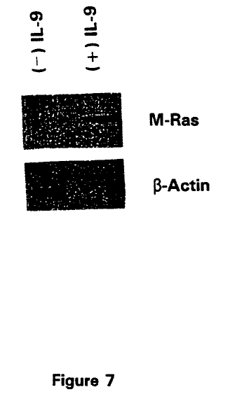

To assess the ability of M-Ras to be induced by the human IL-9 pathway, the autonomously growing human leukemia K562 cell line was assayed for its expression levels of M-Ras in the presence of IL-9. 1×10⁷ log phase K562 cells were harvested and washed three times with phosphate-buffered saline solution and plated in RPMI medium supplemented with 10% fetal bovine serum. Cells were split and supplemented with 50 ng/ml IL-9 for 24 hours. The next day, cells were harvested and total RNA was extracted using the Trizol method as described by the manufacturer (Gibco-BRL). RNA was processed and reverse transcribed into cDNA as described in Example 1. FIG. 7 shows the RT-PCR data demonstrating that M-Ras is induced in K562 cells treated with IL-9 thus demonstrating a conserved IL-9 induction between murine and human IL-9 signaling pathways.

### EXAMPLE 8

### Effect of M-Ras Mutation on Cytokine Dependence of Cells

Mutant Ras molecules have previously been shown to render transformed cells growth factor-independent. To assess the ability of M-Ras to be able to exert a constitutive activity on cellular proliferation and to abrogate the requirement of cytokines in a cytokine-dependent cell line, site directed mutagenesis was used to generate mutant M-Ras proteins to assess their activities. Alignment of M-Ras to the H- and K-Ras proteins permitted identification of potential residues that may result in constitutive activation of M-Ras. Two mutants were prepared at codon 22, substituting a Val or Lys for Gly while another mutant was prepared at codon 71 substituting a Lys for Glu. These substitutions had been previously shown to result in active Ras molecules (FIG. 8). Site directed mutagenesis was performed using the Chameleon double-stranded site directed mutagenesis kit as suggested by the manufacturer (Stratagene). Mutant Ras cDNA was then cloned into the NotI-XbaI cloning site of the mammalian expression vector pEF-BOS which contains a puromycin resistance gene for selection of transfected cells. These expression vectors were then transfected into the IL-3 dependent cell line BaF3 and it was assayed for cytokine-independent growth. BaF3 cells were transfected with the three mutant M-Ras vectors, the wild type M-Ras vector or an empty vector by electroporation using the Gene Pulsar II (Bio-Rad). The settings used were 1500 pF, 74 ohms and 300 volts with 50 μg of sterile DNA. Pooled transfected cells were selected with puromycin (3 μg/ml) and M-Ras expression was determined by Northern blot. All constructs expressed equivalent amounts of exogenous M-Ras. BaF3 cells were then grown in the presence or absence of IL-3 and as shown in FIG. 9, the three constitutive mutant M-Ras transfected cells grew in the absence of IL-3 while the control cells and M-Ras wild-type cells died in the absence of IL-3. This data demonstrates the ability of M-Ras to function in a cell signaling pathway which is essential for cellular proliferation.

### EXAMPLE 9

### M-Ras Transformation of NIH-3T3 Cells

To assess the transforming potential of constitutively active M-Ras, the NIH3T3 foci formation assay was employed (Wigler et al., 1979). NIH3T3 murine fibroblasts were plated 24 hours prior to gene transfer in DMEM medium supplemented with 10% bovine calf serum and glucose. Cells were transfected using the calcium phosphate method with 10 ng expression plasmid containing either wild-type murine M-Ras, M-Ras-1 or activated H-Ras cDNA (Reddy et al., 1982). After 24 hours, cells were washed twice and grown for an additional 21 days in DMEM supplemented with 5% calf serum. Transformed foci were scored after two washes with phosphate-buffered saline, methanol fixing and staining with 2% Giemsa solution for twenty minutes. Foci formation was observed in cells expressing M-Ras-1 or activated H-Ras but not in cells expressing wild-type M-Ras. The fact that aberrant M-Ras activation can cause malignant transformation of NIH3T3 cells suggests that deregulation of M-Ras function may also contribute to spontaneous malignancies.

### EXAMPLE 10

### M-Ras in vivo Signal Transduction Pathway

To characterize the activation pathway triggered by activated M-Ras, the ability of the M-Ras mutants described in Example 8 to activate the mitogen-activated protein kinase (MAPK) pathway or the c-JUN N-terminal Kinase (JNK) pathway was tested using the PathDetect in vivo signal transduction pathway reporting system (Stratagene). A schematic diagram demonstrating the principle of this system is depicted in FIG. 11. HEK293 cells were cotransfected with the following constructs: 1) a luciferase reporter gene controlled by a promoter containing 4 copies of a sequence recognized by the GAL4 yeast transcription factor, 2) a plasmid inducing expression of a fusion protein with the GAL4 DNA binding domain (dbd) fused to the activation domain of Elk (a target of the MAPK pathway) or Jun (a target of the JNK pathway), 3) a plasmid inducing the expression of the gene of interest (M-Ras mutants) or positive controls such as the activated MEK1 (for MAPK pathway) or the activated MEKK (for the JNK pathway). The results of this experiment are shown in FIG. 12 for the MAPK pathway. As expected, significant luciferase activity was detected when cells expressed the GAL4-Elk fusion protein together with activated MEK-1. In addition, M-Ras-1, M-Ras-2 and M-Ras-3 mutants had the same activity, indicating M-Ras induced activation of the MAPK pathway. The luciferase induction by these mutants was blocked by the MAPK inhibitor PD98059 thus demonstrating the role of MAPK and that M-Ras functions upstream from this kinase (FIG. 13). The same results using this reporter assay were obtained in the P815 murine mastocytoma cells, indicating that activation of this pathway is not cell type or species specific. When the same experiment was performed for the JNK pathway, M-Ras mutants did not have any activity suggesting that M-Ras is not involved in this pathway.

### EXAMPLE 11

### Activation of M-Ras Abrogates IL-9 Anti-Apoptotic Activity

Wild type BW5147 cells, a murine T cell lymphoma cell line, undergo apoptosis in the presence of dexamethasone. This effect can be abrogated by the addition of exogenous recombinant IL-9 (Renauld et al., 1995). BW5147 cells were transfected with the constitutively active M-Ras expression vector as described in Example 8. Transfected cells were maintained in selection medium and assayed for resistance to dexamethasone induced apoptosis when grown in the presence of 0.5 μM dexamethasone with or without murine IL-9 for five days. Cell proliferation was measured as described in Example 12. Constitutively active mutants of M-Ras abrogated the requirement for IL-9 for anti-apoptotic activity in BW5147 cells (FIG. 14). This data further supports a role for M-Ras in the IL-9 signaling pathway.

### EXAMPLE 12

### Blocking of IL-9 Induced Proliferation of a Murine T-cell Using MAPK Inhibitors

The murine T-cell line TS2 grows in response to IL-2 and IL-9 in vitro. M-Ras expression was found to be restricted to cells grown in the presence of IL-9 but not IL-2. In addition, the MAPK inhibitor PD98059 has the ability to block M-Ras signaling as described in Example 10. This data suggests that if the same M-Ras pathway was used in all cells that it should be possible to specifically block IL-9 induced proliferation but not IL-2 induced proliferation. Cells which were maintained in medium described in Example 1 plus IL-2, were washed once with phosphate-buffered saline and plated at 3000 cells per well in the presence of 200 U/ml IL-2 or 200 U/ml of IL-9. Cells were grown for three days in the presence of PD98959 (10 μM) and then pulsed for four hours with tritiated thymidine. The cells were then harvested onto glass fiber filters and analyzed by scintillation counting. Results of these experiments are shown in FIG. 15. As shown, cells grown in IL-9 in the presence of the MAPK inhibitor PD98059 were growth inhibited while no inhibitory activity was seen on cells grown in the presence of IL-2 and PD98059. This data demonstrates that the pathway induced by M-Ras is also induced by IL-9 and that blocking this signaling pathway can block IL-9 signaling.

### EXAMPLE 13

### Specific Blocking of M-Ras Signaling in vitro by Small Molecule Inhibitors.

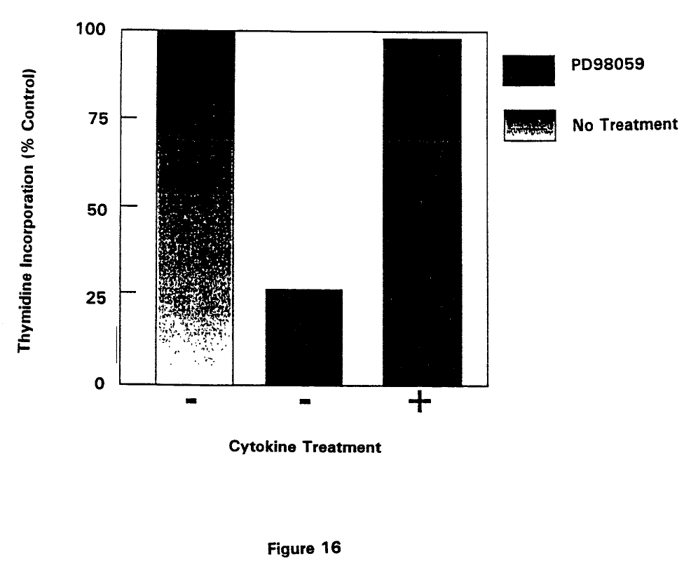

The introduction of constitutively active M-Ras mutants as described in Example 8 abrogates the requirement for IL-3 for proliferation of BaF3 cells even though these cells do not express IL-9 receptor and therefore are unresponsive to IL-9 stimulation. To further demonstrate the specificity of M-Ras signaling which is induced by IL-9, transfected BaF3 cells expressing the constitutively active M-Ras mutant (cytokine-independent) or the wild-type M-Ras cDNA were treated with PD98059 to determine if the inhibition of M-Ras signaling by this compound was cell type specific or if M-Ras uses this pathway in multiple cell types (BaF3, TS2, etc.). Cells were plated at 3000 cells/well in the presence or absence of IL-3 and 10 μm PD98059 and assessed for proliferation as described in Example 12. The wild-type M-Ras was unable to induce proliferation in the absence of IL-3 in BaF3 cells (data not shown, see FIG. 9) while the mutant M-Ras did induce cellular proliferation in the absence of IL-3. The addition of the PD98059 had no activity on blocking IL-3 induced cellular proliferation of M-Ras wild-type transfected cells grown in the presence of IL-3 (FIG. 16, left lane) while the proliferation of cells expressing the mutant M-Ras gene was blocked by this compound (FIG. 16, middle lane). This inhibition was not due to cellular toxicity of the compound since these same cells could proliferate in the presence of the compound when grown with IL-3 (FIG. 16, right lane). This data demonstrates that the M-Ras signaling pathway is not restricted to one cell type and that inhibitors of this pathway in cells expressing M-Ras (IL-9 responsive cells) will block this pathway.

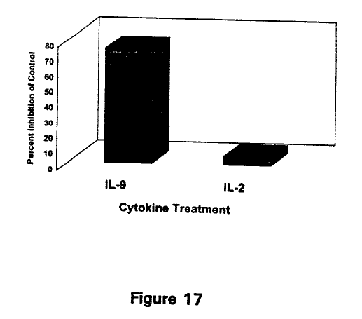

Experiments using an additional MAPK inhibitor, SB202190, also showed the ability to specifically block IL-9 signaling in TS2 cells. As shown in FIG. 17, the presence of SB202190 blocked the ability of TS2 cells to proliferate in response to IL-9 (left bar) but not IL-2 (right bar). The data is shown as percent growth as compared to cytokine only. Interestingly, this compound has been shown to specifically inhibit p38 MAPK and not the ERK1, 2 or 5 family or JNK family of MAPK (Lee et al., 1994; Gallagher et al., 1997). This data suggests that IL-9 signaling occurs through this pathway or a related pathway and suggest proteins involved in signaling via this route(s) should be potential therapeutic targets for blocking IL-9 signaling through M-Ras which in turn should block cellular signaling involved in allergy and asthma.

### EXAMPLE 14

### Blocking IL-9 Induced Cellular Proliferation by a Farnesyl Transferase Inhibitor

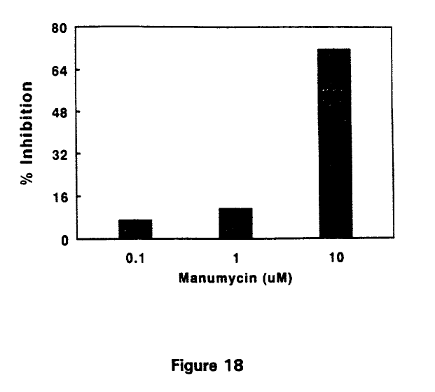

Ras molecules have been previously shown to function after prenlylation via farnesyl transferase and subsequent attachment to the inner cytoplasmic membrane. To test the role of M-Ras in IL-9 signaling, IL-9 responsive cells were treated with the farnesyl transferase inhibitor manumycin A in a proliferation assay to determine if the Ras pathway was utilized for proliferation. TS1-RA3 cells, which are a murine TH2 lymphocyte cell line expressing human IL-9 receptor by a transfected expression construct, were grown in the presence of IL-9 and in the absence or in varying amounts of manumycin A for 4 days. At day 4 cell cultures were assayed by the abacus acid phosphatase assay kit (Clonetech) as suggested by the manufacturer. As shown in FIG. 18, cellular proliferation of TS1-RA3 cells was inhibited up to 76% as compared to untreated cells

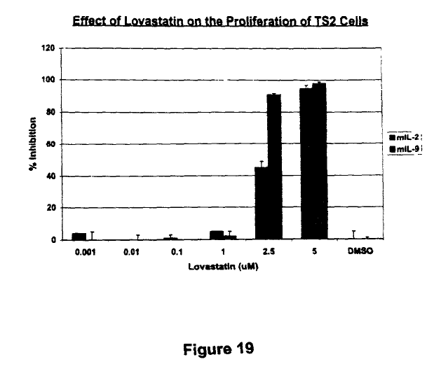

To further test the role of M-Ras in IL-9 signaling, yet another IL-9 responsive cell line was treated with the farnesyl transferase inhibitor lovastatin to determine if the Ras pathway was utilized for proliferation. TS2 cells were washed three times and plated in 96-well plates at a density of 1,000 cells per well. IL-2 or IL-9 was added successively at a concentration of 5 ng/ml together with different concentrations of lovastatin as indicated. DMSO was included as the vehicle control. After three days, the rate of proliferation was determined by ³H-thymidine incorporation (1 mCi per well) and percent of inhibition was obtained from the ratio between treated and untreated cells. As shown in FIG. 19, cellular proliferation of TS2 cells was inhibited up to 95% as compared to untreated cells. This data demonstrates the usefulness of small molecule farnesyl transferase inhibitors in blocking IL-9 induced signaling for cellular proliferation. This data also suggests that, as shown in Examples 10 through 15, small molecules which antagonize the M-Ras pathway may be useful therapeutics for the treatment of asthma-related disorders and certain lymphomas or leukemias.

### EXAMPLE 15

### Blocking of M-Ras Induction by Aminosterols in Murine Splenocytes.

Splenocytes from the DBA2 bronchial hyperresponsive mouse were treated with aminosterol compounds to test for their ability to block the induction of M-Ras in response to mitogens. This group of aminosterols was identified from the liver of the dogfish shark and as a class appear to be antiproliferative. This series of aminosterols was assayed for their ability to inhibit M-Ras expression and TH2 activity in mitogen stimulated splenocytes from the DBA2 mouse. An example of some of these compounds are shown in FIG. 19.

Splenocytes were isolated from naive DBA2 mice by aseptic removal of spleens from anesthetized mice. Spleens were then minced by sterilized scissors and tissue was passed through a sterilized #60 wire mesh sieve. Cells were resuspended in RPMI medium and washed twice in the same medium. Cells were then resuspended in lysis buffer (4.15 g NaCl, 0.5 g KHCO₃, 0.019 g EDTA in 500 ml of dH₂O) to lyse red blood cells. Cells were incubated at 37° C. for 5 minutes and resuspended in RPMI plus heat inactivated 10% fetal bovine serum. Cells were then spun down and resuspended in RPMI growth media (as above) plus 5 ug/ml of concanavalin A. Cell cultures were treated with 10 μg/ml of the aminosterol compounds for 24 hours and were then harvested for RNA isolation using the Trizol method described by the manufacturer (Gibco-BRL).

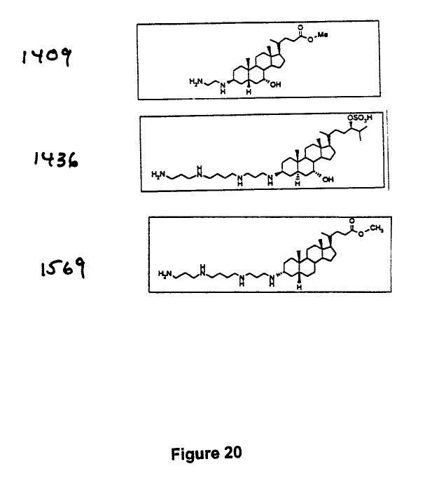

RNA derived from splenocytes treated with aminosterol compounds were reverse transcribed and PCR amplified as described above. FIG. 20 shows the activity of the aminosterols on the expression of M-Ras. The data shows the ability of specific aminosterols, such as 1409, to block the expression of M-Ras in vitro, while similar compounds such as 1436 and 1569 had no effect on expression. This class of compounds will be of use in inhibiting the M-Ras gene expression and its associated activity on inducing the cellular activation of IL-9 responsive cells and their ultimate effect on allergy, asthma and certain lymphomas or leukemias.

### EXAMPLE 16

### Role of M-Ras in Murine Models of Asthma: Airway Response of Unsensitized Animals

DBA2, C57BL6 or B6D2F1 mice, five to six weeks of age, are obtained from the National Cancer Institute or Jackson Laboratories (Bar Harbor ME). IL-9 transgenic mice (Tg5) and their parent strain (FVB) are obtained from the Ludwig Institute (Brussels, Belgium). Animals are housed in high-efficiency particulate filtered air laminar flow hoods in a virus and antigen free facility and allowed free access to pelleted rodent chow and water for three to seven days prior to experimental manipulation. The animal facilities are maintained at 22° C. and the light:dark cycle is automatically controlled (10:14 hour cycle).

Phenotyping and efficacy of pretreatment. To determine the bronchoconstrictor response, respiratory system pressure is measured at the trachea and recorded before and during exposure to the drug. Mice are anesthetized and instrumented as previously described. (Levitt et al., 1988; Levitt et al., 1989; Kleeberger et al., 1990; Levitt et al., 1991; Levitt et al., 1995; Ewart et al., 1995). Airway responsiveness is measured to one or more of the following: 5-hydroxytryptamine, acetylcholine, atracurium or a substance-P analog. A simple and repeatable measure of the change in peak inspiratory pressure following bronchoconstrictor challenge is used which has been termed the Airway Pressure Time Index (APTI) (Levitt et al., 1988; Levitt et al., 1989). The APTI is assessed by the change in peak respiratory pressure integrated from the time of injection until the peak pressure returns to baseline or plateau. The APTI is comparable to airway resistance, however, the APTI includes an additional component related to the recovery from bronchoconstriction.

Prior to sacrifice, whole blood is collected for serum IgE measurements by needle puncture of the inferior vena cava in anesthetized animals. Samples are centrifuged to separate cells and serum is collected and used to measure total IgE levels. Samples not measured immediately are frozen at −20° C.

All IgE serum samples are measured using an ELISA antibody-sandwich assay. Microtiter plates are coated, 50 μl per well, with rat anti-murine IgE antibody (Southern Biotechnology) at a concentration of 2.5 μg/ml in coating buffer of sodium carbonate-sodium bicarbonate with sodium azide. Plates are covered with plastic wrap and incubated at 4° C. for 16 hours. The plates are washed three times with a wash buffer of 0.05% Tween-20 in phosphate-buffered saline, incubating for five minutes for each wash. Blocking of nonspecific binding sites is accomplished by adding 200 μl per well 5% bovine serum albumin in phosphate-buffered saline, covering with plastic wrap and incubating for two hours at 37° C. After washing three times with wash buffer, duplicate 50 pi test samples are added to the wells. Test samples are assayed after being diluted 1:10, 1:50 and 1:100 with 5% bovine serum albumin in wash buffer. In addition to the test samples, a set of IgE standards (PharMingen) at concentrations from 0.8 ng/ml to 200 ng/ml in 5% bovine serum albumin in wash buffer, are assayed to generate a standard curve. A blank of no sample or standard is used to zero the plate reader (background). After adding samples and standards, the plate is covered with plastic wrap and incubated for two hours at room temperature. After washing three times with wash buffer, 50 μl of secondary antibody rat anti-murine IgE-horseradish peroxidase conjugate is added at a concentration of 250 ng/ml in 5% bovine serum albumin in wash buffer. The plate is covered with plastic wrap and incubated two hours at room temperature. After washing three times with wash buffer, 100 μl of the substrate 0.5 mg/ml o-phenylenediamine in 0.1 M citrate buffer is added to every well. After five to ten minutes the reaction is stopped with 50 μl of 12.5% sulfuric acid and absorbance is measured at 490 nm on a MR5000 plate reader (Dynatech). A standard curve is constructed from the standard IgE concentrations with antigen concentration on the x axis (log scale) and absorbance on the y axis (linear scale). The concentration of IgE in the samples is interpolated from the standard curve.

Bronchoalveolar lavage and cellular analysis are preformed as previously described (Kleeberger et al., 1990). Lung histology is carried out after the lungs are removed under anesthesia. Since prior instrumentation may introduce artifact, separate animals are used for these studies. Thus, a small group of animals is treated in parallel exactly the same as the cohort undergoing various pretreatments except these animals are not used for other tests aside from bronchial responsiveness testing. After bronchial responsiveness testing, the lungs are removed and submersed in liquid nitrogen. Cryosectioning and histologic examination is carried out in a manner obvious to those skilled in the art.

Antagonists for the murine M-Ras pathway are used therapeutically to down-regulate the functions of and assess the importance of this pathway to bronchial responsiveness, serum IgE and bronchoalveolar lavage in the sensitized and unsensitized mice. After antagonist pretreatment, baseline bronchial hyperresponsiveness, bronchoalveolar lavage and serum IgE levels relative to Ig matched controls are determined.

### EXAMPLE 17

### Role of M-Ras in Murine Models of Asthma: Airway Response of Sensitized Animals

Animals and handling are essentially as described in Example 16. Sensitization by nasal aspiration of Aspergillus fumigatus antigen is carried out to assess the effect on bronchial hyperresponsiveness, bronchoalveolar lavage and serum IgE. Mice are challenged with Aspergillus or saline intranasally (Monday, Wednesday and Friday for three weeks) and phenotyped 24 hours after the last dose. The effect of pretreatment by antagonists of the M-Ras pathway is used to assess the effect of down-regulating M-Ras in mice.

### EXAMPLE 18

### Lovastatin Inhibition of M-Ras Prenylation

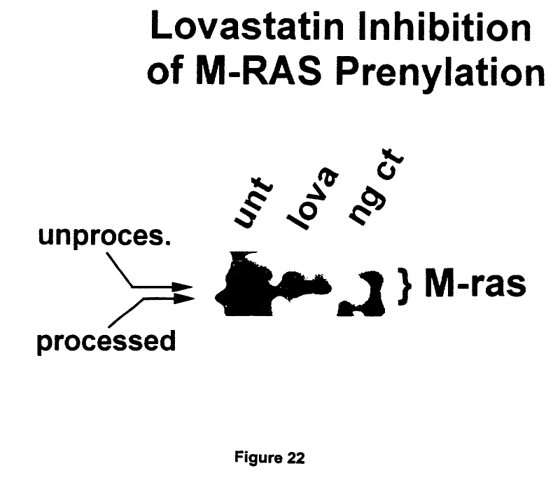

BW5147, a murine thyoma cell line that grows in the absence of any cytokine, was stably transfected with an expression vector containing wild type M-Ras cDNA (pEF-BOS-M-Ras). After two weeks of selection in 3 μg/ml of Puromycin, cells were treated for three days with different compounds previously reported to work as inhibitors of p21 Ras prenylation. Cells were then harvested and proteins were extracted and immunoprecipitated with a rabbit antibody (JAL4) specific for M-Ras. Immunoprecipitates were electrophoretically resolved by SDS-PAGE (18%) and transferred to PVDF membrane. Immunoblots were successively probed with JAL4 antibody (1:1500 dilution) to detect the expression and migratory pattern of M-Ras protein. The non-prenylated form of p21 Ras migrates slower than the fully processed form. As shown in FIG. 22, cells transfected with empty vector pEF-BOS (ng-ct) did not express detectable amounts of M-Ras. In contrast, cells transfected with pEF-BOS-M-Ras overexpressed the M-Ras protein. Additionally, M-Ras protein extracted from cells treated for three days with 4 mg/ml of lovastatin (lova) migrated higher than M-Ras protein extracted from cells left untreated (unt). Since prenylation is necessary for M-Ras activity, this experiment indicates that a fanesyl transferase inhibitor such as lovastatin can down-regulate the activity of M-Ras.

### EXAMPLE 19

### Nucleic Acids which Hybridize to M-Ras

To identify nucleic acid molecules which hybridize to the human or murine M-Ras nucleotide sequences set forth in SEQ ID NOS:1 and 3, hybridization assays are performed using any available methods to control the stringency of hybridization. Hybridization is a function of sequence identity (homology), G+C content of the sequence, buffer salt content, sequence length and duplex melt temperature (Tₘ) among other variables (See Maniatis et al., Molecular Cloning: A Laboratory Manual, Cold Spring Harbor Laboratory Press, 1982).

Hybridization analysis may be performed using genomic DNA or cDNA pools prepared from mRNA from a T lymphocyte cell line according to the procedures of Sambrook et al., Molecular Cloning: A Laboratory Manual, 2nd Edition, Cold Spring Harbor Laboratory Press, 1989, Chapter 9). Briefly, hybridization with nylon membranes is performed in 6× SSC; 0.5% SDS; 100 μg/ml denatured, sonicated salmon sperm DNA and 50% formamide at 42° C. using radiolabeled probe comprising SEQ ID NO:1 and/or SEQ ID NO:3. After hybridization, the filter is washed in 2× SSC and 0.1% SDS, followed by several washes in 0.1× SSC and 0.5% SDS at 37° C. and 0.1× SSC and 0.5% SDS at 68° C. Results are visualized by autoradiography.

Nucleic acid molecules comprising the following sequences hybridize to probe comprising SEQ ID NO:1 under the above conditions: 5′-CCAGACTGGCACAGTTCC-3′ and 5′-TGCTGTAGAAGCCGMGCC-3′.

Nucleic acid molecules comprising the following sequences hybridize to probe comprising SEQ ID NO:3 under the above conditions: 5′-GAATTCAGCGCCATGCGC-3′ and 5′-CCTCACAAGATCACACATTG-3′.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art.

All publications, patents and patent applications herein are incorporated by reference to the same extent as if each individual publication, patent or patent application was specifically and individually indicated to be incorporated by reference.

### REFERENCES

Alexander A G, Barnes N C and Kay A B. Trial of cyclosporin in corticosteroid-dependent chronic severe asthma. Lancet 339, 324-328, 1992.

Altschul S F, Gish W, Miller W, Myers E W and Lipman D J. Basic Local alignment search tool. J. Mol. Biol. 215, 403-410,1990.

Amoah E A and Gelaye S. Biotechnological advances in goat reproduction. J. Anim. Sci. 75, 578-585, 1997.

Bourne H and McCormick F. The GTPase superfamily, conserved structure and molecular mechanism. Nature 349, 117-126, 1991.

Brenin D R, Talamonti M S and lannaccone P M. Transgenic technology: an overview of approaches useful in surgical research. Surg. Oncol. 6, 99-110, 1997.

Burrows B, Sears M R, Flannery E M, Herbison G P and Holdaway M D. Relationship of bronchial responsiveness assessed by methacholine to serum IgE, lung function, symptoms and diagnoses in 11-year-old New Zealand children. J. Allergy Clin. Immunol. 90, 376-385,1992.

Burrows B, Martinez F D, Halonen M, Barbee R A and Cline M G. Association of asthma with serum IgE levels and skin-test reactivity to allergens. New Eng. J. Med. 320, 271-277, 1989.

Clutter A C, Pomp D and Murray J D. Quantitative genetics of transgenic mice: components of phenotypic variation in body weights and weight gains. Genetics 143, 1753-1760, 1996.

Cotton R G. Detection of single base changes in nucleic acids. Biochemical Journal 263(1), 1-10, 1989.

Chomczynski P and Sacchi N. Single-step method of RNA isolation by acid guanidinium thiocyanate-phenol-chloroform extraction. Anal. Biochem. 162,156-159, 1987.

Chu J W and Sharom F J. Glycophorin A interacts with interleukin-2 and inhibits interleukin-2-dependent T-lymphocyte proliferation. Cell. Immunol. 145, 223-239, 1992.

Clifford R D, Pugsley A, Radford M and Holgate S T. Symptoms, atopy and bronchial response to methacholine in parents with asthma and their children. Arch. Dis. Childhood 62, 66-73, 1987.

Doull I, Lawrence S, Watson M, Begishvili T, Beasley R, Lampe F, Holgate S T and Morton N E. Allelic association of makers on chromosome 5q and 11 q with atopy and bronchial hyperresponsiveness. Am. J. Respir. Crit. Care Med. 153, 1280-1284, 1996.

Dugas B, Renauld J C, Pene J, Bonnefoy J, Peti-Frere C, Braquet P, Bousquet J, Van Snick J, Mencia-Huerta J M. lnterleukin-9 potentiates the interieukin-4-induced immunoglobulin (IgG, IgM and IgE) production by normal human B lymphocytes. Eur. J. Immunol. 23, 1687-1692, 1993.

Ewart S, Levitt R C and Mitzner W. Respiratory system mechanics in mice measured by end-inflation occlusion. J. Appl. Phys. 79, 560-566, 1995.

Gallagher T F, Seibel G L, Kassis S, Laydon J T, Blumenthal M J et al. Regulation of stress-induced cytokine production by pyridinylimidazoles; inhibition of CSBP kinase. Bioorg. Med. Chem. 5, 49-54, 1997.

Gergen P J and Weiss K B. The increasing problem of asthma in the United States. Am. Rev. Respir. Dis. 146, 823-824, 1992.

Gergen P J. The association of allergen skin test reactivity and respiratory disease among whites in the U.S. population. Arch. Intem. Med. 151. 487492, 1991.

Halonen M, Stern D, Taussig L M, Wright A, Ray C G and Martinez F D. The predictive relationship between serum IgE levels at birth and subsequent incidences of lower respiratory illnesses and eczema in infants. Am. Rev. Respir. Dis. 146, 666-670, 1992.

Houdebine L M. The production of pharmaceutical proteins from the milk of transgenic animals. Reprod. Nutr. Dev. 35, 609-617, 1995.

Hubank M and Schatz D G. Identifying differences in mRNA expression by representational difference analysis of cDNA. Nucleic Acids Research 22, 5640-5648, 1994.

Johannson S G O, Bennich H H and Berg T. The clinical significance of IgE. Prog. Clin. Immunol. 1, 1-25, 1972.

Kauvar L M. Peptide mimetic drugs: A comment on progress and prospects. Nature Biotechnology 14, 709, 1996.

Kim J H, Jung-Ha H S, Lee H T and Chung K S. Development of a positive method for male stem cell-mediated gene transfer in mouse and pig. Mol. Reprod. Dev. 46, 515-526, 1997.

Kimmelman A, Tolkacheva T, Lorenzi M V, Osada M and Chan A. Identification and characterization of R-ras3: A novel member of the Ras gene family with a non-ubiquitous pattern of tissue distribution. Oncogene 15, 2675-2685, 1997.

Kleeberger S R, Bassett D J, Jakab G J and Levitt R C. A genetic model for evaluation of susceptibility to ozone-induced inflammation. Am. J. Physiol. 258, L313-320, 1990.

Kreitman R J, Puri R K, Leland P et al. Site-specific conjugation to interleukin-4 containing mutated cysteine residues produces interleukin 4-toxin conjugates with improved binding and activity. Biochemistry 33, 11637-11644, 1994.

Lee J C, Laydon J T, McDonnell P C, Gallagher M J, Heys J R, Landvatter S W et al. A protein kinase involved in the regulation of inflammatory cytokine biosynthesis. Nature 372, 739, 1994.

Levitt R C and Ewart S L. Genetic susceptibility to atracurium-induced bronchoconstriction. Am. J. Respir. Crit. Care. Med. 151, 1537-1542, 1995.

Levitt R C. Understanding biological variability in susceptibility to respiratory disease. Pharmacogenetics 1, 94-97,1991.

Levitt R C and Mitzner W. Autosomal recessive inheritance of airway hyperreactivity to 5-hydroxytryptamine. J. AppI. Physiol. 67, 1125-1132, 1989.

Levitt R C, Mitzner W et al. Epression of airway hyperreactivity to acetylcholine as a simple autosomal recessive trait in mice. FASEB J. 2, 2605-2608, 1988.

Lisitsyn N, Lisitsyn N and Wigier M. Cloning the differences between two complex genomes. Science 259, 946-951, 1993.

Louahed J, Kermouni A, Van Snick J and Renauld J C. IL-9 induces expression of granzymes and high affinity IgE receptor in murine T helper clones. J. Immunol. 154, 5061-5070, 1995.

Marsh D G, Meyers D A and Bias W B. The epidemiology and genetics of atopic allergy. New Eng. J. Med. 305, 1551-1559, 1982.

Marshall C J. Ras effectors. Curr. Opin. Cell Biol. 8, 197-204, 1996.

Matsumoto K, Asano T and Endo T. Novel small GTPase M-Ras participates in reorganization of actin cytoskeleton. Oncogene 15, 2409-2417, 1997.

Merz H, Houssiau A, Orscheschek K, Renauld J C, Fliedner A, Herin M, Noel H, Kadin M, Mueller-Hermelink H K and Van Snick J. IL-9 expression in human malignant lymphomas: Unique association with Hodgkins disease and large cell anaplastic lymphoma. Blood 78, 1311-1317, 1991.

Meyers D A, Postma D S, Panhuysen C I M et al. Evidence for a locus regulating total serum IgE levels mapping to chromosome 5. Genomics 23, 464-470, 1994.

Molinoff P et al., Goodman and Gilman's The Pharmacologic Basis of Therapeutics, MacMillan Publishing Company, New York N.Y., 1995.

Morely J. Cyclosporin A in asthma therapy: A pharmacological rationale. J. Autoimmun. 5 Suppl A, 265-269,1992.

Mullins J J and Mullins L J. Transgenesis in nonmurine species. Hypertension 22, 630-633 1993.

Nicolaides N C, Gualdi R, Casadevall C, Manzella L and Calabretta B. Positive autoregulation of c-myb expression via Myb binding sites in the 5′ flanking region of the human c-myb gene. Mol. Cell. Biol. 11, 6166-6176, 1991.

Orita M, Suzuki Y, Sekiya T and Hayashi K. Rapid and sensitive detection of point mutations and DNA polymorphisms using the polymerase chain reaction. Genomics 5, 874-879, 1989.

Ott J. Analysis of human genetic linkage. John Hopkins University Press, Baltimore Md., 1991.

Petters R M. Transgenic livestock as genetic models of human disease. Reprod. Fertil. Dev. 6, 643-645, 1994.

Petit-Frere C, Dugas B, Braquet P, Mencia-Huerta J M. lnterleukin-9 potentiates the interleukin-4-induced IgE and IgGI release from murine B lymphocytes. Immunology 79, 146-151, 1993.

Qian Y, Sebti S M and Hamilton A D. Famesyltransferase as a target for anticancer drug design. Biopolymers 43, 2541,1997.

Quinn T. Low molecular weight GTP-binding proteins and leukocyte signal transduction. J. Leukoc. Biol. 58, 263-276, 1993.

Reddy E P, Reynolds R K, Santos E and Barbacid M. A point mutation is responsible for the acquisition of transforming properties by the T24 human bladder carcinoma oncogene. Nature 300, 149-152, 1982.

Renauld J C, Kermouni A, Vink A, Louahed J and Van Snick J. lnterieukin 9 and its receptor: Involvement in mast cell differentiation and T cell oncogenesis. J. Leukocyte Biol. 57, 353-360, 1995.

Renauld J C, Vink A, Louahed J and Van Snick J. interieukin-9 is a major anti-apoptotic factor for thymic lymphomas. Blood 85, 1300-1305, 1995.

Renauld J C, van der Lugt N, Vink A, van Roon M, Godfraind C, Warnier G, Merz H, Feller A, Berns A and Van Snick J. Thymic lymphomas in interieukin 9 transgenic mice. Oncogene 9, 1327-1332,1994.

Sarkar G, Yoon H-S and Sommer S S. Dideoxy fingerprint (ddF): A rapid and efficient screen for the presence of mutations. Genomics 13, 441-443, 1992.

Schmitt E, Van Brandwijk R, Van Snick J, Siebold B and Rude E. TCGF III/P40 is produced by naive murine CD4+ T cells but is not a general T cell growth factor. Eur. J. Immunol. 19, 2167-2170, 1989.

Schnieke A E, Kind A J, Ritchie W A, Mycock K, Scott A R, Ritchie M, Wilmut I, Colman A and Campbell K H. Human factor IX transgenic sheep produced by transfer of nuclei from transfected fetal fibroblasts. Science 278, 2130-2133, 1997.

Schwengel D, Nouri N, Meyers D and Levitt R C. Linkage mapping of the human thromboxane A2 receptor (TBXA2R) to chromosome 19p13.3 using transcribed 3′ untranslated DNA sequence polymorphisms. Genomics 18, 212-215,1993.

Sears M R, Burrows B, Flannery E M, Herbison G P, Hewitt C J and Holdaway M D. Relation between airway responsiveness and serum IgE in children with asthma and in apparently normal children New Engl. J. Med. 325(15), 1067-1071, 1991.

Self A J and Hall A. Purification of recombinant Rho/RacIG25K from Escherichia coli. Methods Enzymol. 256, 3-10, 1995.

Sheffield V C, Beck J S, Kwitek A E, Sandstrom D W and Stone E M. The sensitivity of single-strand conformation polymorphism analysis for the detection of single base substitutions. Genomics 16, 325-332, 1993.

Simoncsits A, Bristulf J, Tjornhammar M L et al. Deletion mutants of human interleukin 1 beta significantly reduced agonist properties: search for the agonistlantagonist switch in ligands to the interleukin 1 receptors. Cytokine 6, 206-214, 1994.

Stein C A and Cheng Y C. Antisense oligonucleotides as therapeutic agents—is the bullet really magical? Science 20, 261, 1004-1012, 1993.

Vojtek A B, Hollenberg S M and Cooper J A. Mammalian Ras interacts directly with the serine-threonine kinase Raf. Cell 74, 205-214, 1993.

Uyttenhove C R, Simpson R and Van Snick J. Functional and structural characterization of P40: A mouse glycoprotein with T cell growth factor activity. Proc. Natl. Acad. Sci. USA. 85, 6934-6938, 1988.

Vink A, Renauld J C, Wamier G and Van Snick J. Interieukin 9 stimulates in vitro growth of mouse thymic lymphomas. Eur. J. Immunol. 23,1134-1138,1993.

Waskiewicz A and Cooper J. Mitogen and stress response pathway: MAP kinase cascades and phosphatase regulation in mammals and yeast. Curr. Opin. Cell Biol. 7, 798-805, 1995.

Wigler M, Sweet R, Sim G K, Wold B, Pellicer A, Lacy E, Maniatis T, Silverstein S and Axe IR. Transformation of mammalian cells with genes form prokaryotes and eukaryotes. Cell 16, 777-785, 1979.

Zavyalov V P, Navolotskaya E V, Isaev IS et al. Nonapeptide corresponding to the sequence 27-35 of the mature human IL-2 efficiently competes with rIL-2 for binding to thymocyte receptors. Immunol. Lett. 31, 285-288,1992.

What is claimed:

1. A purified and isolated DNA molecule having a nucleotide sequence encoding human M-Ras or functionally equivalent fragments thereof.

2. A purified and isolated DNA molecule having a nucleotide sequence encoding murine M-Ras or functionally equivalent fragments thereof.

3. The purified and isolated DNA molecule of claim 1 or 2, wherein said DNA molecule is genomic.

4. A chemically synthesized DNA molecule having a nucleotide sequence encoding human M-Ras or functionally equivalent fragments thereof.

5. A chemically synthesized DNA molecule having a nucleotide sequence encoding murine M-Ras or functionally equivalent fragments thereof.

6. A purified and isolated RNA molecule having a nucleotide sequence encoding human M-Ras or functionally equivalent fragments thereof.

7. A purified and isolated RNA molecule having a nucleotide sequence encoding murine M-Ras or functionally equivalent fragments thereof.

8. A purified and isolated polypeptide having an amino acid sequence comprising human M-Ras or functionally equivalent fragments thereof.

9. polypeptide having an amino acid sequence comprising murine M-Ras or functionally equivalent fragments thereof.

10. A method of alleviating asthma-related disorders by administering to patients in need of such treatment an equivalent amount of a compound to down-regulate the function of human M-Ras.

11. A method according to claim 10 wherein the compound comprises a farnesyl transferase inhibitor.

12. A method according to claim 11 wherein the farnesyl transferase inhibitor is manumycin A.

13. A method according to claim 11 wherein the farnesyl transferase inhibitor is lovastatin.

14. A method according to claim 10 wherein the compound comprises a geranylgeranyl transferase inhibitor.

15. A method according to claim 10 wherein the compound comprises an aminosterol.

16. A method according to claim 15 wherein the aminosterol is 1409.

17. A method according to claim 10 wherein the compound comprises an inhibitor of the MAPK pathway.

18. A method according to claim 17 wherein the inhibitor of the MAPK pathway is PD98059.

19. A method according to claim 17 wherein the inhibitor of the MAPK pathway is SB202190.

20. A method for detecting or diagnosing susceptibility to asthma-related disorders and certain lymphomas and leukemias associated with elevated levels of M-Ras polypeptide in a human subject comprising the steps of:
(a) measuring the level of M-Ras polypeptide in a biological sample from said human subject; and
(b) comparing the level of M-Ras polypeptide present in normal subjects, wherein an increase in the level of M-Ras polypeptide as compared to normal levels indicates a predisposition to asthma-related disorders and certain lymphomas or leukemias.

21. A method for monitoring a therapeutic treatment of asthma-related disorders or certain lymphomas or leukemias associated with elevated levels of M-Ras polypeptide in a human subject comprising; measuring the levels of M-Ras polypeptide in a series of biologic samples obtained at different time points from said subject undergoing therapeutic treatment wherein a significant decrease in said levels of M-Ras polypeptide indicates a successful therapeutic treatment.

22. A method of treating a tumor by administering to patients in need of such treatment an effective amount of a compound to down-regulate the function of human M-Ras.

23. A method according to claim 22 wherein the compound comprises a farnesyl transferase inhibitor.

24. A method according to claim 23 wherein the farnesyl transferase inhibitor is manumycin A.

25. A method according to claim 23 wherein the farnesyl transferase inhibitor is lovastatin.

26. A method according to claim 22 wherein the compound comprises a geranylgeranyl transferase inhibitor.

27. A method according to claim 22 wherein the compound comprises an aminosterol.

28. A method according to claim 27 wherein the aminosterol is 1409.

29. A method according to claim 22 wherein the compound comprises an inhibitor of the MAPK pathway.

30. A method according to claim 29 wherein the inhibitor of the MAPK pathway is PD98059.

31. A method according to claim 29 wherein the inhibitor of the MAPK pathway is SB202190.

32. A method according to claim 22, wherein the tumor is a T cell lymphoma.

33. A method according to claim 22, wherein the tumor is a T cell leukemia.

34. A method according to claim 22, wherein the tumor is Hodgkin's lymphoma.

35. A method according to claim 22, wherein the tumor is Mycosis fungoides.

36. A method of preparing an antibody specific to an M-Ras polypeptide encoded by the DNA molecule of claim 1 or 2 or fragments thereof comprising the steps of:
(a) conjugating the M-Ras polypeptide or fragments thereof containing at least ten amino acids to a carrier protein;
(b) immunizing a host animal with said M-Ras polypeptide fragment-carrier protein conjugate admixed with an adjuvant; and
(c) obtaining antibody from the immunized host animal.

37. The method of claim 36 wherein the antibody is a monoclonal.

38. A method of quantifying a M-Ras polypeptide of claim 8 or 9 comprising the steps of:
(a) contacting a sample suspected of containing M-Ras polypeptide with an antibody that specifically binds to the M-Ras polypeptide under conditions that allow for the formation of reaction complexes comprising the antibody and M-Ras polypeptide; and
(b) detecting the formation of reaction complexes comprising the antibody and M-Ras polypeptide in the sample, wherein quantitation of the reaction complexes indicates the level of M-Ras polypeptide in the sample.

39. A method for identifying antagonists of M-Ras comprising the steps of:
(a) obtaining a cell line that is responsive to IL-9;
(b) growing said cell line in the presence of IL-9;
(c) comparing the characteristics of IL-9 induction with those obtained with pretreatment with a possible M-Ras antagonist agent; and
(d) selecting those agents for which pretreatment diminished the characteristics.

40. The method according to claim 39 wherein the cell line is taken from the group consisting of: murine TS2 cells, murine BW5147 cells, murine TS1-RA3 cells transfected with the human IL-9 receptor and human K562 cells.

41. A nucleic acid molecule having a nucleotide sequence encoding a M-Ras polypeptide comprising a valine at an amino acid residue corresponding to residue 22 of SEQ ID NO:2 or SEQ ID NO: 4.

42. A nucleic acid molecule having a nucleotide sequence encoding a M-Ras polypeptide comprising a lysine at an amino acid residue corresponding to residue 71 of SEQ ID NO:2 or SEQ ID NO:4.

43. A nucleic acid molecule having a nucleotide sequence encoding a M-Ras poiypeptide comprising a lysine at an amino acid residue corresponding to residue 22 of SEQ ID NO:2 or SEQ ID NO:4.

44. A method for identifying antagonists of M-Ras comprising the steps of:
(a) obtaining a cell line that expresses a constitutively active M-Ras molecule;
(b) treating said cell line with possible M-Ras antagonist agents; and
(c) selecting those agents for which treatment diminished the activity of M-Ras.

45. The method of claim 44 wherein the constitutively active M-Ras is encoded by a nucleic acid molecule of any one of claims 41-43.

46. Antisense DNA comprising the antisense sequence of human M-Ras or active fragments thereof.

47. A method according to claim 10 wherein the compound is the antisense DNA of claim 46.

48. A method according to claim 22 wherein the compound is the antisense DNA of claim 46.

49. An isolated nucleic acid molecule which hybridizes under stringent conditions to a nucleic acid molecule having a sequence complementary to either SEQ ID NO:1 or SEQ ID NO:3.

50. An isolated polypeptide encoded by the nucleic acid molecule of claim 49.

51. The purified and isolated DNA molecule of claim 1 comprising the sequence of SEQ ID NO:3.

52. The purified and isolated DNA molecule of claim 2 comprising the sequence of SEQ ID NO:1
